(19) United States
(12) Patent Application Publication
(10) Pub. No.: US 2026/0250759 A1
(43) Pub. Date: Aug. 27, 2026

(54) METHOD FOR NATIVE STRAND SEQUENCING OF ENRICHED GENOMIC REGIONS

(71) Applicant: NanoHyb, LLC, Heber City, UT (US)

(72) Inventors: Jonathon Hill, Heber City, UT (US); Timothy Jenkins, Heber City, UT (US)

(21) Appl. No.: 19/162,330

(22) PCT Filed: Mar. 7, 2024
(86) PCT No.: PCT/US2024/018903

§ 371 Date: Sep. 4, 2025

Related U.S. Application Data
(60) Provisional application No. 63/450,472, filed on Mar. 7, 2023.
(60) Provisional application No. 63/556,299, filed on Feb. 21, 2024.

(51) Int. Cl. C12Q 1/6869; C12Q 1/25; C12Q 1/48; C12Q 1/6806; C12Q 1/6813; C12Q 1/6855; C40B 70/00

(52) U.S. Cl. CPC C12Q 1/6869; C12Q 1/25; C12Q 1/48; C12Q 1/6806; C12Q 1/6813; C12Q 1/6855; C12Y 207/07; C12Y 605/01001; C40B 70/00; C12Q 2600/154; C12Q 2600/156

(57) ABSTRACT

Provided herein, inter alia, are methods for sequencing a polynucleotide. In addition, provided herein are methods for detecting a base modification in polynucleotide, such as methylation calling, by sequencing only the native strand. The key innovation in this method is the creation of a second strand following hybridization that is configured to only allow adapter ligation to one end. This limits sequencing to the native strand, which is the only strand with potential modifications.

### CROSS-REFERENCE TO RELATED APPLICATIONS

This application is an International Application which claims priority to U.S. Provisional Application Nos. 63/450,472 filed on Mar. 7, 2023, and 63/556,299 filed on Feb. 21, 2024, each of which is incorporated herein by reference in its entirety and for all purposes.

### BACKGROUND

Quantifying the location and frequency of base modifications in the genome, especially methylated cytosines, is an essential tool for studying gene regulation, epigenetic inheritance, and other biological processes, as well as diagnosing disease in the clinic. Current array-based methods for identifying methylated cytosines are expensive, have limited accuracy and dynamic range, and cannot identify other base modifications or determine the type of methylation present. In response, researchers in the lab and the clinic are increasingly moving to reduced representation bisulfite sequencing (RRBS) or reduced representation methylation-sequencing (RRMS). However, RRBS is also technically limited due to the difficulty and inconsistency of enrichment at regions of interest using bisulfite-converted DNA. Additionally, RRMS is often not a realistic alternative as it requires large amounts of sequencing because the whole genome must be sequenced. Thus, improved methods are needed that allow for targeted assessment of modified bases at specific genomic regions in a cost-effective and accurate manner. Ideally, this technique would avoid the two major contributors to assay variance: amplification, and bisulfite (or enzymatic) conversion of unmethylated cytosines. The present disclosure addresses these and other concerns in the art.

### SUMMARY

In an aspect is provided a method for detecting a base modification in a target polynucleotide, the method including: (a) contacting the target polynucleotide with one or more adapters in the presence of a first ligase under conditions promoting ligation of the one or more adapters to the target polynucleotide to create an adapter-target polynucleotide, wherein the one or more adapters include a primer binding site; (b) contacting the adapter-target polynucleotide with one or more tags under conditions promoting hybridization of the one or more tags to the adapter-target polynucleotide to create a tagged adapter-target polynucleotide, wherein the one or more tags are complementary to one or more regions of the target polynucleotide; (c) contacting the tagged adapter-target polynucleotide with a solid support, under conditions promoting hybridization of the one or more tags to the solid support to create a pulldown complex; (d) isolating the pulldown complex; (e) optionally eluting the adapter-target polynucleotide from the pulldown complex; (f) contacting the pulldown complex or eluted adapter-target polynucleotide with a first polymerase under conditions promoting creation of a complementary adapter-target polynucleotide, wherein the complementary adapter-target polynucleotide includes a 3′ protective group or a 5′ protective group; (g) contacting the adapter-target polynucleotide with a sequencing adapter in the presence of a second ligase under conditions promoting ligation of the sequencing adapter to the adapter-target polynucleotide to create a sequencing complex; and (h) sequencing the sequencing complex, wherein the sequencing includes sequencing of the target polynucleotide sequence without sequencing the complementary adapter-target polynucleotide, thereby detecting the base modification in the target polynucleotide.

In another aspect is provided a method for sequencing a target polynucleotide, the method including: (a) contacting the target polynucleotide with one or more adapters in the presence of a first ligase under conditions promoting ligation of the one or more adapters to the target polynucleotide to create an adapter-target polynucleotide, wherein the one or more adapters include a primer binding site; (b) contacting the adapter-target polynucleotide with one or more tags under conditions promoting hybridization of the one or more tags to the adapter-target polynucleotide to create a tagged adapter-target polynucleotide, wherein the one or more tags are complementary to one or more regions of the target polynucleotide; (c) contacting the tagged adapter-target polynucleotide with a solid support, under conditions promoting hybridization of the one or more tags to the solid support to create a pulldown complex; (d) isolating the pulldown complex; (e) optionally eluting the adapter-target polynucleotide from the pulldown complex; (f) contacting the pulldown complex or eluted adapter-target polynucleotide with a first polymerase under conditions promoting creation of a complementary adapter-target polynucleotide, wherein the complementary adapter-target polynucleotide includes a 3′ protective group or a 5′ protective group; (g) contacting the adapter-target polynucleotide with a sequencing adapter in the presence of a second ligase under conditions promoting ligation of the sequencing adapter to the adapter-target polynucleotide to create a sequencing complex; and (h) sequencing the sequencing complex, wherein the sequencing includes sequencing of the target polynucleotide sequence without sequencing the complementary adapter-target polynucleotide, thereby sequencing the target polynucleotide, optionally wherein the target polynucleotide includes one or more base modifications.

In another aspect is provided a method for quantifying a base modification in a target polynucleotide, the method including: (a) contacting the target polynucleotide with one or more adapters in the presence of a first ligase under conditions promoting ligation of the one or more adapters to the target polynucleotide to create an adapter-target polynucleotide, wherein the one or more adapters include a primer binding site; (b) contacting the adapter-target polynucleotide with one or more tags under conditions promoting hybridization of the one or more tags to the adapter-target polynucleotide to create a tagged adapter-target polynucleotide, wherein the one or more tags are complementary to one or more regions of the target polynucleotide; (c) contacting the tagged adapter-target polynucleotide with a solid support, under conditions promoting hybridization of the one or more tags to the solid support to create a pulldown complex; (d) isolating the pulldown complex; (e) optionally eluting the adapter-target polynucleotide from the pulldown complex; (f) contacting the pulldown complex or eluted adapter-target polynucleotide with a first polymerase under conditions promoting creation of a complementary adapter-target polynucleotide, wherein the complementary adapter-target polynucleotide includes a 3′ protective group or a 5′ protective group; (g) contacting the adapter-target polynucleotide with a sequencing adapter in the presence of a second ligase under conditions promoting ligation of the sequencing adapter to the adapter-target polynucleotide to create a sequencing complex; and (h) sequencing the sequencing complex, wherein the sequencing includes sequencing of the target polynucleotide sequence without sequencing the complementary adapter-target polynucleotide, thereby quantifying the base modification in the target polynucleotide.

In another aspect is provided a method for detecting a plurality of base modifications in a plurality of target polynucleotides, the method including: (a) contacting the plurality of target polynucleotides with one or more adapters in the presence of a first ligase under conditions promoting ligation of the one or more adapters to the plurality of target polynucleotides to create a plurality of adapter-target polynucleotides, wherein the one or more adapters include a primer binding site; (b) contacting the plurality of adapter-target polynucleotides with one or more tags under conditions promoting hybridization of the one or more tags to the plurality of adapter-target polynucleotides to create a plurality of tagged adapter-target polynucleotides, wherein the one or more tags are complementary to one or more regions of the plurality of target polynucleotides; (c) contacting the plurality of tagged adapter-target polynucleotides with a solid support, under conditions promoting hybridization of the one or more tags to the solid support to create a plurality of pulldown complexes; (d) isolating the plurality of pulldown complexes; (e) optionally eluting the plurality of adapter-target polynucleotides from the plurality of pulldown complexes; (f) contacting the plurality of pulldown complexes or plurality of eluted adapter-target polynucleotides with a first polymerase under conditions promoting creation of a plurality of complementary adapter-target polynucleotides, wherein each of the complementary adapter-target polynucleotides include a 3′ protective group or a 5′ protective group; (g) contacting the plurality of adapter-target polynucleotides with a plurality of sequencing adapters in the presence of a second ligase under conditions promoting ligation of the plurality of sequencing adapters to the plurality of adapter-target polynucleotides to create a plurality of sequencing complexes; and (h) sequencing the plurality of sequencing complexes, wherein the sequencing includes sequencing of the plurality of target polynucleotide sequences without sequencing the plurality of complementary adapter-target polynucleotides, thereby detecting a plurality of base modifications in the plurality of target polynucleotides.

In another aspect is provided a method for sequencing a plurality of target polynucleotides, the method including: (a) contacting the plurality of target polynucleotides with one or more adapters in the presence of a first ligase under conditions promoting ligation of the one or more adapters to the plurality of target polynucleotides to create a plurality of adapter-target polynucleotides, wherein the one or more adapters include a primer binding site; (b) contacting the plurality of adapter-target polynucleotides with one or more tags under conditions promoting hybridization of the one or more tags to the plurality of adapter-target polynucleotides to create a plurality of tagged adapter-target polynucleotides, wherein the one or more tags are complementary to one or more regions of the plurality of target polynucleotides; (c) contacting the plurality of tagged adapter-target polynucleotides with a solid support, under conditions promoting hybridization of the one or more tags to the solid support to create a plurality of pulldown complexes; (d) isolating the plurality of pulldown complexes; (e) optionally eluting the plurality of adapter-target polynucleotides from the plurality of pulldown complexes; (f) contacting the plurality of pulldown complexes or plurality of eluted adapter-target polynucleotides with a first polymerase under conditions promoting creation of a plurality of complementary adapter-target polynucleotides, wherein each of the complementary adapter-target polynucleotides include a 3′ protective group or a 5′ protective group; (g) contacting the plurality of adapter-target polynucleotides with a plurality of sequencing adapters in the presence of a second ligase under conditions promoting ligation of the plurality of sequencing adapters to the plurality of adapter-target polynucleotides to create a plurality of sequencing complexes; and (h) sequencing the plurality of sequencing complexes, wherein the sequencing includes sequencing of the plurality of target polynucleotide sequences without sequencing the plurality of complementary adapter-target polynucleotides, thereby sequencing the plurality of target polynucleotides, optionally wherein the plurality of target polynucleotides include one or more base modifications.

In another aspect is provided a method for detecting a plurality of base modifications in a target polynucleotide, the method including: (a) contacting the target polynucleotide with one or more adapters in the presence of a first ligase under conditions promoting ligation of the one or more adapters to the target polynucleotide to create an adapter-target polynucleotide, wherein the one or more adapters include a primer binding site; (b) contacting the adapter-target polynucleotide with one or more tags under conditions promoting hybridization of the one or more tags to the adapter-target polynucleotide to create a tagged adapter-target polynucleotide, wherein the one or more tags are complementary to one or more regions of the target polynucleotide; (c) contacting the tagged adapter-target polynucleotide with a solid support, under conditions promoting hybridization of the one or more tags to the solid support to create a pulldown complex; (d) isolating the pulldown complex; (e) optionally eluting the adapter-target polynucleotide from the pulldown complex; (f) contacting the pulldown complex or eluted adapter-target polynucleotide with a first polymerase under conditions promoting creation of a complementary adapter-target polynucleotide, wherein the complementary adapter-target polynucleotide includes a 3′ protective group or a 5′ protective group; (g) contacting the adapter-target polynucleotide with a sequencing adapter in the presence of a second ligase under conditions promoting ligation of the sequencing adapter to the adapter-target polynucleotide to create a sequencing complex; and (h) sequencing the sequencing complex, wherein the sequencing includes sequencing of the target polynucleotide sequence without sequencing the complementary adapter-target polynucleotide, thereby detecting the plurality of base modifications in the target polynucleotide.

In another aspect is provided a method for detecting a base modification in a plurality of target polynucleotides, the method including: (a) contacting the plurality of target polynucleotides with one or more adapters in the presence of a first ligase under conditions promoting ligation of the one or more adapters to the plurality of target polynucleotides to create a plurality of adapter-target polynucleotides, wherein the one or more adapters include a primer binding site; (b) contacting the plurality of adapter-target polynucleotides with one or more tags under conditions promoting hybridization of the one or more tags to the plurality of adapter-target polynucleotides to create a plurality of tagged adapter-target polynucleotides, wherein the one or more tags are complementary to one or more regions of the plurality of target polynucleotides; (c) contacting the plurality of tagged adapter-target polynucleotides with a solid support, under conditions promoting hybridization of the one or more tags to the solid support to create a plurality of pulldown complexes; (d) isolating the plurality of pulldown complexes; (e) optionally eluting the plurality of adapter-target polynucleotides from the plurality of pulldown complexes; (f) contacting the plurality of pulldown complexes or plurality of eluted adapter-target polynucleotides with a first polymerase under conditions promoting creation of a plurality of complementary adapter-target polynucleotides, wherein each of the complementary adapter-target polynucleotides include a 3′ protective group or a 5′ protective group; (g) contacting the plurality of adapter-target polynucleotides with a plurality of sequencing adapters in the presence of a second ligase under conditions promoting ligation of the plurality of sequencing adapters to the plurality of adapter-target polynucleotides to create a plurality of sequencing complexes; and (h) sequencing the plurality of sequencing complexes, wherein the sequencing includes sequencing of the plurality of target polynucleotide sequences without sequencing the plurality of complementary adapter-target polynucleotides, thereby detecting a base modifications in the plurality of target polynucleotides.

### BRIEF DESCRIPTION OF THE DRAWINGS

FIG. 1 shows an exemplary schematic of an enrichment method for native methylation calling. Step 1: Adapters containing a primer binding site (orange) and a barcode (purple) are ligated to the input DNA. Step 2: Biotinylated DNA polynucleotides complimentary to the region(s) of interest are hybridized to the DNA input. Step 3: Streptavidin beads are used to pulldown and isolate the desired regions. Step 4: The DNA is eluted from the beads. Step 5: A complimentary strand is synthesized using a primer to the 3′ end of the DNA and a polymerase. Polymerase activity also leaves an A at the other end. Step 6: A sequencing adapter is added to the end of the DNA opposite the primer site. This results in only the native strand being sequenced.

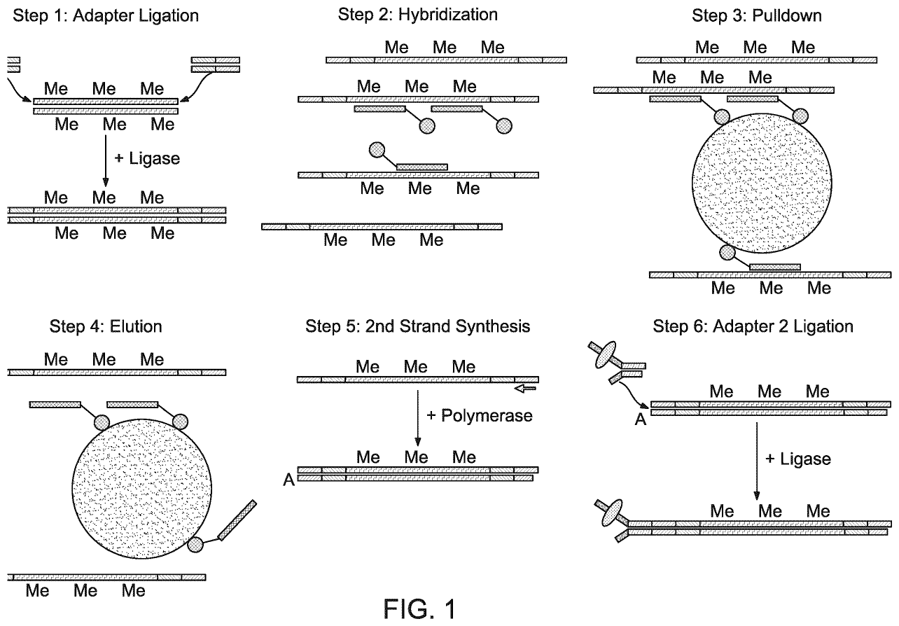

FIGS. 2A-2B show experimental results using native methylation calling following enrichment. FIG. 2A: Histogram of methylation frequency at enriched sites, showing that the full range of methylation is identified. FIG. 2B: Correlation between microarray methylation frequency with Nanohyb enriched regions following Nanopore sequencing. Correlation is similar to levels seen between whole genome methylation sequencing and array.

FIGS. 3A-3B show results from an analysis of methylation patterns in sperm.

FIG. 4 shows Copy Number Variations (CNVs) detected using the method described herein.

### DETAILED DESCRIPTION

After reading this description it will become apparent to one skilled in the art how to implement the present disclosure in various alternative embodiments and alternative applications. However, all the various embodiments of the present invention will not be described herein. It will be understood that the embodiments presented here are presented by way of an example only, and not limitation. As such, this detailed description of various alternative embodiments should not be construed to limit the scope or breadth of the present disclosure as set forth herein.

Before the present technology is disclosed and described, it is to be understood that the aspects described below are not limited to specific compositions, methods of preparing such compositions, or uses thereof as such may, of course, vary. It is also to be understood that the terminology used herein is for the purpose of describing particular aspects only and is not intended to be limiting.

The detailed description divided into various sections only for the reader's convenience and disclosure found in any section may be combined with that in another section. Titles or subtitles may be used in the specification for the convenience of a reader, which are not intended to influence the scope of the present disclosure.

### Definitions

Unless defined otherwise, all technical and scientific terms used herein have the same meaning as commonly understood by one of ordinary skill in the art to which this disclosure belongs. In this specification and in the claims that follow, reference will be made to a number of terms that shall be defined to have the following meanings:

The terminology used herein is for the purpose of describing particular embodiments only and is not intended to be limiting. As used herein, the singular forms “a”, “an” and “the” are intended to include the plural forms as well, unless the context clearly indicates otherwise.

“Optional” or “optionally” means that the subsequently described event or circumstance can or cannot occur, and that the description includes instances where the event or circumstance occurs and instances where it does not.

The term “about” when used before a numerical designation, e.g., temperature, time, amount, concentration, and such other, including a range, indicates approximations which may vary by (+) or (−) 10%, 5%, 1%, or any subrange or subvalue there between. Preferably, the term “about” when used with regard to an amount means that the amount may vary by +/−10%.

As used herein, the term “each,” when used in reference to a collection of items, is intended to identify an individual item in the collection but does not necessarily refer to every item in the collection. Exceptions can occur if explicit disclosure or context clearly dictates otherwise.

“Comprising” or “comprises” is intended to mean that the compositions and methods include the recited elements, but not excluding others. “Consisting essentially of” when used to define compositions and methods, shall mean excluding other elements of any essential significance to the combination for the stated purpose. Thus, a composition consisting essentially of the elements as defined herein would not exclude other materials or steps that do not materially affect the basic and novel characteristic(s) of the claimed invention. “Consisting of” shall mean excluding more than trace elements of other ingredients and substantial method steps. Embodiments defined by each of these transition terms are within the scope of this disclosure.

As may be used herein, the terms “nucleic acid,” “nucleic acid molecule,” “nucleic acid oligomer,” “oligonucleotide,” “nucleic acid sequence,” “nucleic acid fragment” and “polynucleotide” are used interchangeably and are intended to include, but are not limited to, a polymeric form of nucleotides covalently linked together that may have various lengths, either deoxyribonucleotides or ribonucleotides, or analogs, derivatives or modifications thereof. Different polynucleotides may have different three-dimensional structures, and may perform various functions, known or unknown. Non-limiting examples of polynucleotides include a gene, a gene fragment, an exon, an intron, intergenic DNA (including, without limitation, heterochromatic DNA), messenger RNA (mRNA), transfer RNA, ribosomal RNA, a ribozyme, cDNA, a recombinant polynucleotide, a branched polynucleotide, a plasmid, a vector, isolated DNA of a sequence, isolated RNA of a sequence, a nucleic acid probe, and a primer. Polynucleotides useful in the methods of the disclosure may comprise natural nucleic acid sequences and variants thereof, artificial nucleic acid sequences, or a combination of such sequences.

A polynucleotide is typically composed of a specific sequence of four nucleotide bases: adenine (A); cytosine (C); guanine (G); and thymine (T) (uracil (U) for thymine (T) when the polynucleotide is RNA). Thus, the term “polynucleotide sequence” is the alphabetical representation of a polynucleotide molecule; alternatively, the term may be applied to the polynucleotide molecule itself. This alphabetical representation can be input into databases in a computer having a central processing unit and used for bioinformatics applications such as functional genomics and homology searching. Polynucleotides may optionally include one or more non-standard nucleotide(s), nucleotide analog(s) and/or modified nucleotides.

“Nucleic acid” refers to nucleotides (e.g., deoxyribonucleotides or ribonucleotides) and polymers thereof in either single-, double- or multiple-stranded form, or complements thereof; or nucleosides (e.g., deoxyribonucleosides or ribonucleosides). In embodiments, “nucleic acid” does not include nucleosides. The terms “polynucleotide,” “oligonucleotide,” “oligo” or the like refer, in the usual and customary sense, to a linear sequence of nucleotides. The term “nucleoside” refers, in the usual and customary sense, to a glycosylamine including a nucleobase and a five-carbon sugar (ribose or deoxyribose). Non limiting examples, of nucleosides include, cytidine, uridine, adenosine, guanosine, thymidine and inosine. The term “nucleotide” refers, in the usual and customary sense, to a single unit of a polynucleotide, i.e., a monomer. Nucleotides can be ribonucleotides, deoxyribonucleotides, or modified versions thereof. Examples of polynucleotides contemplated herein include single and double stranded DNA, single and double stranded RNA, and hybrid molecules having mixtures of single and double stranded DNA and RNA. Examples of nucleic acid, e.g., polynucleotides contemplated herein include any types of RNA, e.g., mRNA, siRNA, miRNA, and guide RNA and any types of DNA, genomic DNA, plasmid DNA, and minicircle DNA, and any fragments thereof. The term “duplex” in the context of polynucleotides refers, in the usual and customary sense, to double strandedness. Nucleic acids can be linear or branched. For example, nucleic acids can be a linear chain of nucleotides or the nucleic acids can be branched, e.g., such that the nucleic acids comprise one or more arms or branches of nucleotides. Optionally, the branched nucleic acids are repetitively branched to form higher ordered structures such as dendrimers and the like.

The term “nucleotide” according to the present disclosure particularly relates to ribonucleotides, 2′-deoxyribonucleotides or 2′,3′-dideoxyribonucleotides.

The term “nucleobase” refers to either native or non-native purine or pyrimidine bases. Nucleobases include adenine, cytosine, guanine, thymine, uracil, hypoxanthine, xanthine, 7-deaza-adenine and 7-deazaguanine, inosine.

As used herein, the phrase “dNTP” means 2′-deoxynucleotidetriphosphate, where the nucleotide comprises a native or non-native nucleobase.

As used herein, the term “double-stranded,” when used in reference to a polynucleotide, means that some or all of the nucleotides between complementary strands of a polynucleotide are hydrogen bonded together to form a partial or complete double helix. A partially double stranded polynucleotide can have at least 10%, 25%, 50%, 60%, 70%, 80%, 90% or 95% of its nucleotides hydrogen bonded to a complementary nucleotide.

A single-stranded polynucleotide refers to a polynucleotide that has few to no hydrogen bonds with another polynucleotide such that a double helix is not formed or is unstable under a given set of hybridization conditions.

A “polymerase” is generally an enzyme that catalyzes the reaction between 3′-OH and 5′-triphosphate in nucleotides, oligomers, and their analogs to form nucleic acid polymers. Polymerases include, but are not limited to, DNA-dependent DNA polymerases, RNA-dependent DNA polymerases, template-independent DNA polymerase, T7 DNA polymerase, T3 DNA polymerase, T4 DNA polymerase, DNA polymerase 1, Klenow fragment, Thermophilus aquaticus DNA polymerase, Tth DNA polymerase, Phusion DNA Polymerase, SuperFi DNA Polymerase, Vent DNA polymerase, Deep Vent DNA polymerase, Bst DNA Polymerase Large Fragment, Stoeffel Fragment, 9° N DNA Polymerase, Pfu DNA Polymerase, Tfl DNA Polymerase, Phi29 Polymerase, Tli DNA polymerase, eukaryotic DNA polymerase beta, telomerase, KOD HiFi, KOD1 DNA polymerase, Q-beta replicase, terminal transferase (TdT), AMV reverse transcriptase, M-MLV reverse transcriptase, Phi6 reverse transcriptase, HIV-1 reverse transcriptase, Thermo Sequenase (Thermo Fisher Scientific). These polymerases include wild-type, mutant isoforms, chimeric forms, and genetically engineered variants such as exo-polymerases and other mutants, e.g., that tolerate modified nucleotides and incorporate them into a strand of nucleic acid.

As used herein, the term “base modification” refers to a nucleotide that is modified through the addition of chemical groups or substituted with a non-natural nucleotide or a non-natural nucleoside. In embodiments, the base modification includes methylation. In embodiments, the base modification includes a 5-methylcytosine (5mC), an N 6-methyladenine (6 mA), a 7-methylguanine (m7G), or 2′-O-methylation (2′-O-Methyl). In embodiments, the base modification includes oxidation of a methylated nucleotide. In embodiments, the base modification includes a 5-hydroxymethylcytosine (5hmC), a 5-formylcytosine (5fC), a 5-carboxylcytosine (5caC), a 5-hydroxymethyluracil (5hmU), a 5formyluracil (5hmU), or a β-D-glucosyl-hydroxymethyluracil (base J). In embodiments, the base modification includes a bromodeoxyuridine (BrdU).

As used herein, the term “methylation” refers to the process by which methyl groups are added to a nucleotide. In embodiments, methylation includes addition of methyl groups to adenine, cytosine, or guanine. In embodiments, methylation includes 2′-O-methylation (2′-O-Methyl).

As used herein, the term “non-natural nucleotide” refers to nucleotide analogs, synthetic nucleotides, and nucleotide mimetics which are not found in nature. In embodiments, a non-natural nucleotide can replace a natural nucleotide found in a polynucleotide. In embodiments, a non-natural nucleotide can substitute a natural nucleotide found in a polynucleotide.

As used herein, the term “non-natural nucleoside” refers to nucleoside analogs, synthetic nucleosides, and nucleoside mimetics which are not found in nature. In embodiments, a non-natural nucleoside can replace a natural nucleoside found in a polynucleotide. In embodiments, a non-natural nucleoside can substitute a natural nucleoside found in a polynucleotide. In embodiments, the non-natural nucleoside is bromodeoxyuridine (BrdU).

As used herein, the term “target polynucleotide” refers to a polynucleotide that is selected for sequencing. For example, and without limitation, the target polynucleotide may be a polynucleotide having or expected to have a base modification. In embodiments, the target polynucleotide may be a polynucleotide having or expected to have a base modification in a given cell type. In embodiments, the target polynucleotide may be a genomic polynucleotide. In embodiments, the target polynucleotide may be genomic DNA. In embodiments, the target polynucleotide may be part of the genome of a cell. In embodiments, the target polynucleotide may be a fragment of the genome of a cell. In embodiments, the target polynucleotide may be DNA from a particular cell type. In embodiments, the target polynucleotide may be used to identify a cell type. In embodiments, the target polynucleotide may be from any compartment within a cell. In embodiments, the target polynucleotide may be a nuclear polynucleotide. In embodiments, the target polynucleotide may be nuclear DNA. In embodiments, the target polynucleotide may be a mitochondrial polynucleotide. In embodiments, the target polynucleotide may be mitochondrial DNA (mDNA). In embodiments, the target polynucleotide may be part of the mitochondrial DNA of a cell. In embodiments, the target polynucleotide may be a mitochondrial DNA fragment. In embodiments, the target polynucleotide may be a DNA fragment. In embodiments, the target polynucleotide may be chromosomal DNA or a fragment thereof. In embodiments, the target polynucleotide may be outside a cell. In embodiments, the target polynucleotide may be cell-free DNA (cfDNA). In embodiments, the target polynucleotide may be included in a biological sample. In embodiments, the target polynucleotide may be isolated from a biological sample. In embodiments, the target polynucleotide may be DNA from an animal. In embodiments, the target polynucleotide may be DNA from a vertebrate animal. In embodiments, the target polynucleotide may be DNA from a mammal. In embodiments, the target polynucleotide may be DNA from a human. In embodiments, the target polynucleotide may be DNA from a rodent. In embodiments, the target polynucleotide may be DNA from a mouse. In embodiments, the target polynucleotide may be DNA from a rat. In embodiments, the target polynucleotide may be DNA from an invertebrate animal. In embodiments, the target polynucleotide may be DNA from a plant. In embodiments, the target polynucleotide may be DNA from a pathogen. In embodiments, the target polynucleotide may be DNA from a virus. In embodiments, the target polynucleotide may be DNA from bacteria. In embodiments, the target polynucleotide may be DNA from a parasite. In embodiments, the target polynucleotide may be DNA from an archaeon.

As used herein, the term “adapter” refers generally to any oligonucleotide that can be added, for example, ligated, to a nucleic acid molecule, thereby generating nucleic acid products that can be sequenced on a sequencing platform. In some embodiments, adapters include two complementary oligonucleotides forming a double-stranded structure. In embodiments, the adapter is an oligonucleotide that can be ligated to the end of a target polynucleotide.

As used herein, the term “ligation” refers to the process of joining two biomolecules (e.g., oligonucleotides, polynucleotides) by forming a chemical bond between their ends. In embodiments, the chemical bond is a non-covalent chemical bond. In embodiments, ligation is catalyzed by a ligase.

As used herein, the term “ligase” refer to an enzyme that catalyzes the ligation of two biomolecules (e.g., oligonucleotides, polynucleotides) by forming a new chemical bond.

As used herein, the term “adapter-target polynucleotide” refers to a target polynucleotide which has one or more adapters ligated to an end (or both ends) of the target polynucleotide.

As used herein, the term “primer binding site” refers to a nucleotide sequence of a polynucleotide to which a primer binds and serves as the site of initiation for replication of the polynucleotide.

As used herein, the term “complementary adapter-target polynucleotide” refers to a polynucleotide that is complementary to and hybridizes to the adapter-target polynucleotide. In embodiments, the complementary adapter-targe polynucleotide includes a 3′ protective group. In embodiments, the complementary adapter-targe polynucleotide includes a 5′ protective group. In embodiments, the complementary adapter-target polynucleotide is complementary to all or substantially all of the adapter-target polynucleotide. In embodiments, the complementary adapter-target polynucleotide is complementary to a portion of the adapter-target polynucleotide.

As used herein, the term “3′ protective group” refers to a moiety on the end of a polynucleotide to prevent sequencing of the polynucleotide. In embodiments, the 3′ protective group prevents sequencing of the complementary adapter-target polynucleotide. In embodiments, the 3′ protective group prevents ligation of a sequencing adapter to the complementary adapter-target polynucleotide. In embodiments, the 3′ protective group includes a nucleotide overhang or a 3′ phosphate. In embodiments, the 3′ protective group includes a nucleotide overhang. In embodiments, the 3′ protective group includes a 3′ phosphate.

As used herein, the term “5′ protective group” refers to a moiety on the 5′ end of a polynucleotide to prevent sequencing of the polynucleotide. In embodiments, the 5′ protective group prevents sequencing of the complementary adapter-target polynucleotide. In embodiments, the 5′ protective group prevents ligation of a sequencing adapter to the complementary adapter-target polynucleotide. In embodiments, the 5′ protective group includes an aldehyde, an amine, or a thiol. In embodiments, the 5′ protective group includes an aldehyde. In embodiments, the 5′ protective group includes an amine. In embodiments, the 5′ protective group includes a thiol. In embodiments, the 5′ protective group includes a 5′ aldehyde. In embodiments, the 5′ protective group includes a 5′ amine. In embodiments, the 5′ protective group includes a 5′ thiol.

As used herein, the term nucleotide overhang refers to a sequence of unpaired nucleotides at the end of a polynucleotide.

As used herein, the term “complementary” or “substantially complementary” refers to the hybridization, base pairing, or the formation of a duplex between nucleotides or nucleic acids. For example, complementarity exists between the two strands of a double-stranded DNA molecule or between an oligonucleotide primer and a primer binding site on a single-stranded nucleic acid when a nucleotide (e.g., RNA or DNA) or a sequence of nucleotides is capable of base pairing with a respective cognate nucleotide or cognate sequence of nucleotides. As described herein and commonly known in the art the complementary (matching) nucleotide of adenosine (A) is thymidine (T) and the complementary (matching) nucleotide of guanosine (G) is cytosine (C). Thus, a complement may include a sequence of nucleotides that base pair with corresponding complementary nucleotides of a second nucleic acid sequence. The nucleotides of a complement may partially or completely match the nucleotides of the second nucleic acid sequence. Where the nucleotides of the complement completely match each nucleotide of the second nucleic acid sequence, the complement forms base pairs with each nucleotide of the second nucleic acid sequence. Where the nucleotides of the complement partially match the nucleotides of the second nucleic acid sequence only some of the nucleotides of the complement form base pairs with nucleotides of the second nucleic acid sequence. Examples of complementary sequences include coding and non-coding sequences, wherein the non-coding sequence contains complementary nucleotides to the coding sequence and thus forms the complement of the coding sequence. A further example of complementary sequences are sense and antisense sequences, wherein the sense sequence contains complementary nucleotides to the antisense sequence and thus forms the complement of the antisense sequence. “Duplex” means at least two oligonucleotides and/or polynucleotides that are fully or partially complementary undergo Watson-Crick type base pairing among all or most of their nucleotides so that a stable complex is formed. In embodiments, a first template polynucleotide and a second template polynucleotide of an overlapping cluster are not substantially complementary (e.g., are at least 50%, 75%, 90%, or more non-complementary to each other).

As described herein, the complementarity of sequences may be partial, in which only some of the nucleic acids match according to base pairing, or complete, where all the nucleic acids match according to base pairing. Thus, two sequences that are complementary to each other, may have a specified percentage of nucleotides that complement one another (e.g., about 60%, preferably 65%, 70%, 75%, 80%, 85%, 90%, 91%, 92%, 93%, 94%, 95%, 96%, 97%, 98%, 99%, or higher complementarity over a specified region). In embodiments, two sequences are complementary when they are completely complementary, having 100% complementarity. In embodiments, sequences in a pair of complementary sequences form portions of a single polynucleotide with non-base-pairing nucleotides (e.g., as in a hairpin or loop structure, with or without an overhang) or portions of separate polynucleotides. In embodiments, one or both sequences in a pair of complementary sequences form portions of longer polynucleotides, which may or may not include additional regions of complementarity.

As used herein, the term “contacting” is used in accordance with its plain ordinary meaning and refers to the process of allowing at least two distinct species (e.g., chemical compounds including biomolecules or cells) to become sufficiently proximal to react, interact or physically touch. However, the resulting reaction product can be produced directly from a reaction between the added reagents or from an intermediate from one or more of the added reagents that can be produced in the reaction mixture. The term “contacting” may include allowing two species to react, interact, or physically touch, wherein the two species may be a compound, nucleic acid, a protein, or enzyme (e.g., a DNA polymerase).

As used herein, the term “barcode” refers to a known nucleic acid sequence that allows some feature of a nucleic acid with which the barcode is associated to be identified. In some embodiments, the feature of the nucleic acid to be identified is the sample or source from which the nucleic acid is derived. By way of example only, some embodiments described herein describe the addition of multiple barcodes (e.g., 2, 3, 4, 5, 6, or more) to the nucleic acids of interest in a single cell present in a population of cells. The unique combination of barcodes added to the nucleic acids of each individual cells can advantageously enable the identification of the cell from which the tagged nucleic acid of interest was derived. In some embodiments, barcodes are at least 3, 4, 5, 6, 7, 8, 9, 10, 11, 12, 13, 14, 15, or more nucleotides in length. In some embodiments, barcodes are shorter than 10, 9, 8, 7, 6, 5, or 4 nucleotides in length. In some embodiments, barcodes associated with some nucleic acids are of a different length than barcodes associated with other nucleic acids. In general, barcodes are of sufficient length and comprise sequences that are sufficiently different to allow the identification of samples based on barcodes with which they are associated. In some embodiments, a barcode and the sample source with which it is associated can be identified accurately after the mutation, insertion, or deletion of one or more nucleotides in the barcode sequence, such as the mutation, insertion, or deletion. In some embodiments, each barcode in a plurality of barcodes differs from every other barcode in the plurality at two or more nucleotide positions, such as at 2, 3, 4, 5, 6, 7, 8, 9, 10, or more positions. In some embodiments, one or more adaptors comprise(s) at least one of a plurality of barcode sequences. In some embodiments, methods of the technology further comprise identifying the sample or source from which a target nucleic acid is derived based on a barcode sequence to which the target nucleic acid is joined. In some embodiments, methods of the technology further comprise identifying the target nucleic acid based on a barcode sequence to which the target nucleic acid is joined. Some embodiments of the method further comprise identifying a source or sample of the target nucleotide sequence by determining a barcode nucleotide sequence. Some embodiments of the method further comprise molecular counting applications (e.g., digital barcode enumeration and/or binning) to determine expression levels or copy number status of desired targets. In general, a barcode may comprise a nucleic acid sequence that when joined to a target nucleic acid serves as an identifier of the sample from which the target polynucleotide was derived.

As used herein, the term “primer” or “extension primer” refers to an oligonucleotide, whether occurring naturally or produced synthetically, that is capable of acting as a point of initiation of nucleic acid synthesis when placed under appropriate conditions, e.g., in the presence of nucleotide triphosphates and a polymerase enzyme (for example, a thermostable polymerase enzyme) in an appropriate buffer (“buffer” includes appropriate pH, ionic strength, cofactors, etc.) and at a suitable temperature. The primer may be, in some embodiments, single-stranded for maximum efficiency in amplification but may alternatively be double-stranded. If double-stranded, the primer is first treated to separate its strands before being used to prepare extension products. In some embodiments, the primer is an oligodeoxyribonucleotide. The primer must be sufficiently long to prime the synthesis of extension products. The exact lengths of the primers will depend on many factors, including temperature, source of primer, the polymerase enzyme (for example, whether it is thermostable), and use of the method.

Nucleic acids can include nonspecific sequences. As used herein, the term “nonspecific sequence” refers to a nucleic acid sequence that contains a series of residues that are not designed to be complementary to or are only partially complementary to any other nucleic acid sequence. By way of example, a nonspecific nucleic acid sequence is a sequence of nucleic acid residues that does not function as an inhibitory nucleic acid when contacted with a cell or organism.

The term “complement,” as used herein, refers to a nucleotide (e.g., RNA or DNA) or a sequence of nucleotides capable of base pairing with a complementary nucleotide or sequence of nucleotides. As described herein and commonly known in the art the complementary (matching) nucleotide of adenosine is thymidine and the complementary (matching) nucleotide of guanosine is cytosine. Thus, a complement may include a sequence of nucleotides that base pair with corresponding complementary nucleotides of a second nucleic acid sequence. The nucleotides of a complement may partially or completely match the nucleotides of the second nucleic acid sequence. Where the nucleotides of the complement completely match each nucleotide of the second nucleic acid sequence, the complement forms base pairs with each nucleotide of the second nucleic acid sequence. Where the nucleotides of the complement partially match the nucleotides of the second nucleic acid sequence only some of the nucleotides of the complement form base pairs with nucleotides of the second nucleic acid sequence. Examples of complementary sequences include coding and a non-coding sequences, wherein the non-coding sequence contains complementary nucleotides to the coding sequence and thus forms the complement of the coding sequence. A further example of complementary sequences are sense and antisense sequences, wherein the sense sequence contains complementary nucleotides to the antisense sequence and thus forms the complement of the antisense sequence.

As described herein the complementarity of sequences may be partial, in which only some of the nucleic acids match according to base pairing, or complete, where all the nucleic acids match according to base pairing. Thus, two sequences that are complementary to each other, may have a specified percentage of nucleotides that are the same (i.e., about 60% identity, preferably 65%, 70%, 75%, 80%, 85%, 90%, 91%, 92%, 93%, 94%, 95%, 96%, 97%, 98%, 99%, or higher identity over a specified region).

As used herein, the terms “library”, “RNA library” or “DNA library” or “library of DNA molecules” are used in accordance with their plain ordinary meaning and refer to a collection or a population of similarly sized nucleic acid fragments with known adapter sequences (e.g., known adapters attached to the 5′ and 3′ ends of each of the fragments). In embodiments, the library includes a plurality of nucleic acid fragments including one or more adapter sequences. In embodiments, the library includes circular nucleic acid templates. Libraries are typically prepared from input RNA, DNA, or cDNA and are processed by fragmentation, size selection, end-repair, adapter ligation, amplification, and purification. Alternative amplification-free (i.e., PCR free) methods for preparing a library of molecules include shearing input polynucleotides, size selecting and ligating adapters. A library may correspond to a single sample or a single origin. Multiple libraries, each with their own unique adapter sequences, may be pooled and sequenced in the same sequencing run using the methods described herein.

As used herein, the term “extension” or “elongation” is used in accordance with their plain and ordinary meanings and refer to synthesis by a polymerase of a new polynucleotide strand (e.g., an “extension strand”) complementary to a template strand by adding free nucleotides (e.g., dNTPs) from a reaction mixture that are complementary to the template in a 5′-to-3′ direction, including condensing a 5′-phosphate group of a dNTPs with a 3′-hydroxy group at the end of the nascent (elongating) DNA strand.

As used herein, the terms “sequencing”, “sequence determination”, “determining a nucleotide sequence”, and the like include determination of a partial or complete sequence information, including the identification, ordering, or locations of the nucleotides that comprise the polynucleotide being sequenced, and inclusive of the physical processes for generating such sequence information. That is, the term includes sequence comparisons, consensus sequence determination, contig assembly, fingerprinting, and like levels of information about a target polynucleotide, as well as the express identification and ordering of nucleotides in a target polynucleotide. The term also includes the determination of the identification, ordering, and locations of one, two, or three of the four types of nucleotides within a target polynucleotide. In some embodiments, a sequencing process described herein comprises contacting a template and an annealed primer with a suitable polymerase under conditions suitable for polymerase extension and/or sequencing.

As used herein, the term “sequencing read” is used in accordance with its plain and ordinary meaning and refers to an inferred sequence of nucleotide bases (or nucleotide base probabilities) corresponding to all or part of a single polynucleotide fragment. A sequencing read may include 10, 20, 30, 40, 50, 60, 70, 80, 90, 100, 150, 200, 250, or more nucleotide bases.

The term “gene” means the segment of DNA involved in producing a protein; it includes regions preceding and following the coding region (leader and trailer) as well as intervening sequences (introns) between individual coding segments (exons). The leader, the trailer as well as the introns include regulatory elements that are necessary during the transcription and the translation of a gene. Further, a “protein gene product” is a protein expressed from a particular gene.

As used herein, the term “hybridize” or “specifically hybridize” refers to a process where two complementary nucleic acid strands anneal to each other under appropriately stringent conditions. Hybridizations are typically and preferably conducted with oligonucleotides. The terms “annealing” and “hybridization” are used interchangeably to mean the formation of a stable duplex. The propensity for hybridization between nucleic acids depends on the temperature and ionic strength of their milieu, the length of the nucleic acids and the degree of complementarity. The effect of these parameters on hybridization is described in, for example, Sambrook J., Fritsch E. F., Maniatis T., Molecular cloning: a laboratory manual, Cold Spring Harbor Laboratory Press, New York (1989). Those skilled in the art understand how to estimate and adjust the stringency of hybridization conditions such that sequences having at least a desired level of complementarity will stably hybridize, while those having lower complementarity will not. As used herein, hybridization of a primer, or of a DNA extension product, respectively, is extendable by creation of a phosphodiester bond with an available nucleotide or nucleotide analogue capable of forming a phosphodiester bond, therewith.

For specific proteins described herein, the named protein includes any of the protein's naturally occurring forms, variants or homologs that maintain the protein transcription factor activity (e.g., within at least 50%, 80%, 90%, 95%, 96%, 97%, 98%, 99% or 100% activity compared to the native protein). In some embodiments, variants or homologs have at least 90%, 95%, 96%, 97%, 98%, 99% or 100% amino acid sequence identity across the whole sequence or a portion of the sequence (e.g., a 50, 100, 150 or 200 continuous amino acid portion) compared to a naturally occurring form. In other embodiments, the protein is the protein as identified by its NCBI sequence reference. In other embodiments, the protein is the protein as identified by its NCBI sequence reference, homolog or functional fragment thereof.

As used herein, the term “library”, when used in reference to nucleic acids, is intended to mean a collection of nucleic acids having different chemical compositions (e.g., different sequence, different length, etc.). Typically, the nucleic acids in a library will be different species having a common feature or characteristic of a genus or class, but otherwise differing in some way. For example, a library can include nucleic acid species that differ in nucleotide sequence, but that are similar with respect to having a sugar-phosphate backbone. A library can be created using techniques known in the art. Nucleic acids exemplified herein can include nucleic acids obtained from any source, including for example, digestion of a genome (e.g., a human genome) or a mixture of genomes. In another example, nucleic acids can be those obtained from metagenomic studies of a particular environment or ecosystem. The term also includes artificially created nucleic acid libraries such as DNA libraries.

The terms “click chemistry” and “click reaction” are used interchangeably herein and are intended to be consistent with their use in the art. Generally, click chemistry reactions are fast (e.g., quick to completion of reaction), simple, easily purified, and regiospecific. Click chemistry includes reactions such as, but not limited to, copper catalyzed azide-alkyne cycloaddition (CuAAC); strain-promoted azide-alkyne cycloaddition (SPAAC) also known as copper-free click chemistry; strain-promoted alkyne-nitrone cycloaddition (SPANC); alkyne hydrothiolation; and alkene hydrothiolation. Click chemistry using copper as a catalyst often includes a Cu(I) stabilizing ligand that is labile. Without being bound by any particular theory, the ligand can stabilize or protect the Cu(I) ion from oxidizing from the reactive Cu(I) to Cu(II) and can also act as a proton acceptor reducing or eliminating requirement of a base in the reaction. Click chemistry between polynucleotides can in some embodiments, be assisted by using a moiety that brings the two reacting partners in close enough proximity to react.

As used herein, and unless otherwise specified, the term “azide” or “azido” refers to N3, or —N═N+═N—, or —N—N+° N.

As used herein, “labels” are chemical or biochemical moieties useful for labeling a nucleic acid. “Labels” include, for example, fluorescent agents, chemiluminescent agents, affinity agents, blocking groups, chromogenic agents, quenching agents, radionucleotides, enzymes, substrates, cofactors, inhibitors, nanoparticles, magnetic particles, and other moieties known in the art. Labels are capable of generating a measurable signal and may be covalently or non-covalently joined to an oligonucleotide or nucleotide. In some examples, an oligonucleotide or a portion of an oligonucleotide of oligonucleotide-tethered nucleotide as disclosed herein may serve as a label.

As used herein, the term “tagged adapter-target polynucleotide” refers to an adapter-target polynucleotide that has one or more tags bound to it.

As used herein, the term “tag” refers to a biomolecule capable of binding an adapter-target polynucleotide. In embodiments, the tag includes a polynucleotide sequence which is complementary to a region of the target polynucleotide. In embodiments, the tag is capable of binding to the complementary region of the target polynucleotide. In embodiments, the tag includes one half of a cognate binding pair.

As used herein, the term “solid support” refers to an insoluble, chemically inert object that has a solid surface. Examples of a solid support provided herein include, but are not limited to, a bead, a column, a chip, a well, an array, a microfluidic channel, a resin, a particle, a microparticle, or a nanoparticle. In embodiments, the surface of the solid support can have a smooth, a porous, or a granular surface. In embodiments, the solid support includes one half of a cognate pair. In embodiments, the surface of the solid support includes one half of a cognate pair.

As used herein, the term “cognate binding pair” refers to a pair of biomolecules which non-covalently bind each other. In embodiments, the cognate binding pair comprises a capture moiety and a binding moiety. In some embodiments, the solid support comprises a capture moiety. In some embodiments, the tag comprises a binding moiety. In embodiments, the non-covalent binding of the cognate pair forms a cognate capture complex. In some embodiments, the non-covalent binding of the cognate pair forms a pulldown complex. In embodiments, the cognate pair includes the binding pairs of streptavidin and biotin, maltose and maltose binding protein, glutathione and glutathione S-transferase, chitin and chitin binding protein, an aptamer and its antigen, SpyCatcher and SpyTag, or an antibody and its antigen. In embodiments, the cognate pair includes the binding pair of streptavidin and biotin. In embodiments, the cognate pair includes the binding pair of maltose and maltose binding protein. In embodiments, the cognate pair includes the binding pair of glutathione and glutathione S-transferase. In embodiments, the cognate pair includes the binding pair of chitin and chitin binding protein. In embodiments, the cognate pair includes the binding pair of an aptamer and its antigen. In embodiments, the cognate pair includes the binding pair of SpyCatcher and SpyTag. In embodiments, the cognate pair includes the binding pair of an antibody and its antigen.

As used herein, the term “pulldown complex” refers to the complex formed when a binding moiety hybridizes to its corresponding capture moiety, thereby binding a tagged adapter-target polynucleotide to a solid support. In embodiments, the pulldown complex includes a tagged adapter-target polynucleotide and a solid support.

As used herein, the term “sequencing adapter” refers to a synthetic oligonucleotide or synthetic polynucleotide that flanks either side or flanks both sides of a polynucleotide sequence that will be sequenced. In embodiments, the sequencing adapter includes a flow cell binding sequence. In embodiments, the sequencing adapter includes a sequencer primer binding site. In embodiments, the sequencing adapter includes an index region or a barcode region. In embodiments, the sequencing adapter includes a motor protein binding site.

As used herein, the term “motor protein binding site” refers to an oligonucleotide sequence that allows for the binding of a motor protein. In embodiments, the motor protein is a polypeptide that unwinds double-stranded polynucleotides for sequencing of a single-stranded polynucleotide. In embodiments, the motor protein includes helicase activity.

As used herein, the term “flow cell binding sequence” refers to an oligonucleotide sequence that is complementary to the oligonucleotides attached to the sequencing flow cell. In embodiments, the flow cell binding sequence binds the polynucleotide sequence of interest to the flow cell.

As used herein, the term “sequencer primer binding site” refers to an oligonucleotide sequence allows for the binding of a sequencing primer. In embodiments, a sequencing primer is a synthetic oligonucleotide that recruits a polymerase to bind and extend the oligo synthesis.

As used herein, the term “index region” or “barcode region” refers to region of a sequencing adapter that allows for the individual sequencing adapter to be identified. In embodiments, one or more sequencing adapters include one or more index regions which allow for multiplexing.

As used herein, the term “sequencing complex” refers to a complex formed by the ligation of one or more sequencing adapters to an adapter-target polynucleotide. In embodiments, the sequencing complex includes one or more sequencing adapters and an adapter-target polynucleotide.

As used herein, the term “sequencing” refers to a process of determining the sequence of a biopolymer. In embodiments, the biopolymer is a polynucleotide. In embodiments, the sequencing does not include bisulfite sequencing. In embodiments, the sequencing does not include whole-genome sequencing.

As used herein, the term “bisulfite sequencing” refers to a sequencing method used to determine the methylation pattern of a polynucleotide. In embodiments, bisulfite sequencing includes bisulfite conversion. In embodiments, the bisulfite conversion includes treating a polynucleotide with bisulfite before sequencing. In embodiments, the bisulfite treatment introduces specific changes in the sequence of the polynucleotide. In embodiments, the bisulfite treatment converts cytosine residues to uracil. In embodiments, the bisulfite treatment does not convert 5-methylcytosine residues to uracil. In embodiments, the bisulfite sequencing includes reduced representation bisulfite sequencing (RRBS).

As used herein, the term “reduced representation bisulfite sequencing” or “RRBS” refers to a sequencing technique that analyzes the genome-wide methylation pattern on a single nucleotide level. In embodiments, RRBS includes restriction enzymes and bisulfite sequencing to enrich for areas of the genome with a high CpG content. In embodiments, RRBS does not target any promoter region.

As used herein, the term “reduced methylation sequencing” or “RRMS” refers to a sequencing technique that analyzes the genome-wide methylation pattern without bisulfite conversion. In embodiments, RRMS includes adaptive sampling to enrich for regions of interest. In embodiments, adaptive sampling deletes off-target regions during sequencing.

As used herein, the term “whole genome sequencing” or “WGS” refers to a sequencing technique used to determine the entirety or nearly the entirety of the nucleotide sequence of an organism's genome.

As used herein, the term “cell-free DNA” or “cfDNA” refers to freely circulating (e.g., not contained within a cell) DNA fragments found in a biological sample.

Biological sample” or “sample” refer to materials obtained from or derived from a subject or patient. A biological sample includes sections of tissues such as biopsy and autopsy samples, and frozen sections taken for histological purposes. Such samples include bodily fluids such as blood and blood fractions or products (e.g., serum, plasma, platelets, red blood cells, and the like), sputum, tissue, cultured cells (e.g., primary cultures, explants, and transformed cells) stool, urine, semen, synovial fluid, joint tissue, synovial tissue, synoviocytes, fibroblast-like synoviocytes, macrophage-like synoviocytes, immune cells, hematopoietic cells, fibroblasts, macrophages, T cells, etc. A biological sample is typically obtained from a eukaryotic organism, such as a mammal such as a primate e.g., chimpanzee or human; cow; horse; goat; pig; dog; cat; a rodent, e.g., guinea pig, rat, mouse; rabbit; or a bird; reptile; or fish. In embodiments the biological sample is obtained from a vertebrate animal. In embodiments, the biological sample is obtained from an invertebrate animal. In embodiments, the biological sample is obtained from a bacterium. In embodiments, the biological sample is obtained from a virus. In embodiments, the biological sample is obtained from a plant. In embodiments, the biologial sample is obtained from an archaea.

As used herein, the term “barcode” refers to a known nucleic acid sequence that allows some feature of a polynucleotide with which the barcode is associated to be identified. In some embodiments, the feature of the polynucleotide to be identified is the sample or source from which the polynucleotide is derived. In some embodiments, barcodes are at least 2, 3, 4, 5, 6, 7, 8, 9, 10, 11, 12, 13, 14, 15, or more nucleotides in length. In some embodiments, barcodes are shorter than 10, 9, 8, 7, 6, 5, or 4 nucleotides in length. In some embodiments, barcodes associated with some polynucleotides are of a different length than barcodes associated with other polynucleotides. In general, barcodes are of sufficient length and comprise sequences that are sufficiently different to allow the identification of samples based on barcodes with which they are associated. In some embodiments, each barcode in a plurality of barcodes differs from every other barcode in the plurality at two or more nucleotide positions, such as at 2, 3, 4, 5, 6, 7, 8, 9, 10, or more positions. In some embodiments, one or more adaptors comprise(s) at least one of a plurality of barcode sequences. In some embodiments, methods of the technology further comprise identifying the polynucleotide based on a barcode sequence to which the polynucleotide is joined. In general, a barcode may comprise an oligonucleotide sequence that when joined to a polynucleotide serves as an identifier of the sample from which the polynucleotide was derived.

### Methods

Provided herein are methods for sequencing target polynucleotides. In some embodiments, the target polynucleotide is enriched without amplification. In some embodiments, the sequencing comprises detecting or identifying a base modification in the target polynucleotide.

In some embodiments, the method includes a first step for ligating adapters to a target polynucleotide. In some embodiments, the first step includes ligating one or more adapters to a target polynucleotide to form an adapter-target polynucleotide. In some embodiments, the one or more adapters include a primer binding site.

In some embodiments, the method includes a second step for hybridizing tags to the target polynucleotide. In some embodiments, the second step includes hybridizing one or more tags to the target polynucleotide to form a tagged adapter-target polynucleotide. In some embodiments, the one or more tags include a polynucleotide sequence complementary to the target polynucleotide. In some embodiments, the one or more tags include one half of a cognate pair. In some embodiments, the one or more tags include a binding moiety.

In some embodiments, the method includes a third step including formation of a pulldown complex. In some embodiments, the formation of the pulldown complex includes the binding of a cognate pair. In some embodiments, the formation of the pulldown complex includes the binding of a binding moiety to a capture moiety. In some embodiments, the pulldown complex includes the tagged adapter-target polynucleotide bound to a solid support. In some embodiments, the solid support includes one half of a cognate pair. In some embodiments, the solid support includes a capture moiety.

In some embodiments, the method includes a fourth step for pulldown the target polynucleotide. In some embodiments, the fourth step includes isolating the pulldown complex. In some embodiments, the isolation of the pulldown complex enriches the target polynucleotide. In embodiments, the isolation of the pulldown complex may include exposing the pulldown complex to a magnet. In embodiments, the isolation of the pulldown complex may include exposing the pulldown complex to a magnet for about 1 minute. In embodiments, the isolation of the pulldown complex may include one or more exposures to a magnet. In embodiments, the isolation of the pulldown complex may include one or more exposures of the pulldown complex to a magnet, wherein each exposure is about 1 minute. In embodiments, the isolation of the pulldown complex may include two exposures of the pulldown complex to a magnet, wherein each exposure is about 1 minute. In embodiments, the isolation of the pulldown complex may include three exposures of the pulldown complex to a magnet, wherein each exposure is about 1 minute. In embodiments, the isolation of the pulldown complex may include incubating the pulldown complex with one or more wash buffers. In embodiments, the isolation of the pulldown complex may include incubating the pulldown complex with one wash buffer. In embodiments, the isolation of the pulldown complex may include incubating the pulldown complex with two wash buffers. In embodiments, the pulldown complex is incubated with a wash buffer for 5 minutes. In embodiments, the pulldown complex is incubated with one or more wash buffers, wherein each incubation occurs for 5 minutes. In embodiments, the pulldown complex is incubated with a wash buffer at 68° C. In embodiments, the pulldown complex is incubated with a wash buffer at 48° C.

In some embodiments, the method optionally includes a fifth step for eluting the target polynucleotide from the pulldown complex. In some embodiments, the optional fifth step includes eluting the adapter-target polynucleotide from the pulldown complex. In embodiments, the elution of the adapter-target polynucleotide from the pulldown complex may include incubation for about 5 minutes. In embodiments, the elution of the adapter-target polynucleotide from the pulldown complex may include incubation at room temperature (RT).

In some embodiments, the method includes a sixth step for second strand synthesis. In some embodiments, the sixth step includes synthesizing a complementary adapter-target polynucleotide. In some embodiments, the pulldown complex or eluted adapter-target polynucleotide is contacted with a polymerase under conditions that promote creation of a complementary adapter-target polynucleotide. In some embodiments, the complementary adapter-target polynucleotide includes a 3′ protective group. In some embodiments, the 3′ protective group prevents sequencing of the synthesized complementary adapter-target polynucleotide. In some embodiments, the synthesized complementary adapter-target polynucleotide includes a 5′ protective group. In some embodiments, the 5′ protective group prevents sequencing of the synthesized complementary adapter-target polynucleotide.

In some embodiments, the method includes a seventh step for formation of a sequencing complex. In some embodiments, the seventh step includes ligating a sequencing adapter to the adapter-target polynucleotide to form a sequencing complex. In some embodiments, the 3′ protective group prevents ligation of the sequencing adapter to the complementary adapter-target polynucleotide. In some embodiments, the 5′ protective group prevents ligation of the sequencing adapter to the complementary adapter-target polynucleotide. In some embodiments, a primer without a 5′ is used to prevent ligation of the sequencing adapter to the complementary adapter-target polynucleotide.

In some embodiments, the method includes an eighth step including sequencing the sequencing complex. In some embodiments, the complementary adapter-target polynucleotide is not sequenced.

In another aspect is provided a method for detecting a base modification in a target polynucleotide, the method including: (a) contacting the target polynucleotide with one or more adapters in the presence of a first ligase under conditions promoting ligation of the one or more adapters to the target polynucleotide to create an adapter-target polynucleotide, wherein the one or more adapters include a primer binding site; (b) contacting the adapter-target polynucleotide with one or more tags under conditions promoting hybridization of the one or more tags to the adapter-target polynucleotide to create a tagged adapter-target polynucleotide, wherein the one or more tags are complementary to one or more regions of the target polynucleotide; (c) contacting the tagged adapter-target polynucleotide with a solid support, under conditions promoting hybridization of the one or more tags to the solid support to create a pulldown complex; (d) isolating the pulldown complex; (e) optionally eluting the adapter-target polynucleotide from the pulldown complex; (f) contacting the pulldown complex or eluted adapter-target polynucleotide with a first polymerase under conditions promoting creation of a complementary adapter-target polynucleotide, wherein the complementary adapter-target polynucleotide includes a 3′ protective group or a 5′ protective group; (g) contacting the adapter-target polynucleotide with a sequencing adapter in the presence of a second ligase under conditions promoting ligation of the sequencing adapter to the adapter-target polynucleotide to create a sequencing complex; and (h) sequencing the sequencing complex, wherein the sequencing includes sequencing of the target polynucleotide sequence without sequencing the complementary adapter-target polynucleotide, thereby detecting the base modification in the target polynucleotide.

In another aspect is provided a method for sequencing a target polynucleotide, the method including: (a) contacting the target polynucleotide with one or more adapters in the presence of a first ligase under conditions promoting ligation of the one or more adapters to the target polynucleotide to create an adapter-target polynucleotide, wherein the one or more adapters include a primer binding site; (b) contacting the adapter-target polynucleotide with one or more tags under conditions promoting hybridization of the one or more tags to the adapter-target polynucleotide to create a tagged adapter-target polynucleotide, wherein the one or more tags are complementary to one or more regions of the target polynucleotide; (c) contacting the tagged adapter-target polynucleotide with a solid support, under conditions promoting hybridization of the one or more tags to the solid support to create a pulldown complex; (d) isolating the pulldown complex; (e) optionally eluting the adapter-target polynucleotide from the pulldown complex; (f) contacting the pulldown complex or eluted adapter-target polynucleotide with a first polymerase under conditions promoting creation of a complementary adapter-target polynucleotide, wherein the complementary adapter-target polynucleotide includes a 3′ protective group or a 5′ protective group; (g) contacting the adapter-target polynucleotide with a sequencing adapter in the presence of a second ligase under conditions promoting ligation of the sequencing adapter to the adapter-target polynucleotide to create a sequencing complex; and (h) sequencing the sequencing complex, wherein the sequencing includes sequencing of the target polynucleotide sequence without sequencing the complementary adapter-target polynucleotide, thereby sequencing the target polynucleotide, optionally wherein the target polynucleotide includes one or more base modifications.

In an aspect is provided a method for quantifying a base modification in a target polynucleotide, the method including: (a) contacting the target polynucleotide with one or more adapters in the presence of a first ligase under conditions promoting ligation of the one or more adapters to the target polynucleotide to create an adapter-target polynucleotide, wherein the one or more adapters include a primer binding site; (b) contacting the adapter-target polynucleotide with one or more tags under conditions promoting hybridization of the one or more tags to the adapter-target polynucleotide to create a tagged adapter-target polynucleotide, wherein the one or more tags are complementary to one or more regions of the target polynucleotide; (c) contacting the tagged adapter-target polynucleotide with a solid support, under conditions promoting hybridization of the one or more tags to the solid support to create a pulldown complex; (d) isolating the pulldown complex; (e) optionally eluting the adapter-target polynucleotide from the pulldown complex; (f) contacting the pulldown complex or eluted adapter-target polynucleotide with a first polymerase under conditions promoting creation of a complementary adapter-target polynucleotide, wherein the complementary adapter-target polynucleotide includes a 3′ protective group or a 5′ protective group; (g) contacting the adapter-target polynucleotide with a sequencing adapter in the presence of a second ligase under conditions promoting ligation of the sequencing adapter to the adapter-target polynucleotide to create a sequencing complex; and (h) sequencing the sequencing complex, wherein the sequencing includes sequencing of the target polynucleotide sequence without sequencing the complementary adapter-target polynucleotide, thereby quantifying the base modification in the target polynucleotide.

In another aspect is provided a method for detecting a plurality of base modifications in a plurality of target polynucleotides, the method including: (a) contacting the plurality of target polynucleotides with one or more adapters in the presence of a first ligase under conditions promoting ligation of the one or more adapters to the plurality of target polynucleotides to create a plurality of adapter-target polynucleotides, wherein the one or more adapters include a primer binding site; (b) contacting the plurality of adapter-target polynucleotides with one or more tags under conditions promoting hybridization of the one or more tags to the plurality of adapter-target polynucleotides to create a plurality of tagged adapter-target polynucleotides, wherein the one or more tags are complementary to one or more regions of the plurality of target polynucleotides; (c) contacting the plurality of tagged adapter-target polynucleotides with a solid support, under conditions promoting hybridization of the one or more tags to the solid support to create a plurality of pulldown complexes; (d) isolating the plurality of pulldown complexes; (e) optionally eluting the plurality of adapter-target polynucleotides from the plurality of pulldown complexes; (f) contacting the plurality of pulldown complexes or plurality of eluted adapter-target polynucleotides with a first polymerase under conditions promoting creation of a plurality of complementary adapter-target polynucleotides, wherein each of the complementary adapter-target polynucleotides include a 3′ protective group or a 5′ protective group; (g) contacting the plurality of adapter-target polynucleotides with a plurality of sequencing adapters in the presence of a second ligase under conditions promoting ligation of the plurality of sequencing adapters to the plurality of adapter-target polynucleotides to create a plurality of sequencing complexes; and (h) sequencing the plurality of sequencing complexes, wherein the sequencing includes sequencing of the plurality of target polynucleotide sequences without sequencing the plurality of complementary adapter-target polynucleotides, thereby detecting a plurality of base modifications in the plurality of target polynucleotides.

In another aspect is provided a method for sequencing a plurality of target polynucleotides, the method including: (a) contacting the plurality of target polynucleotides with one or more adapters in the presence of a first ligase under conditions promoting ligation of the one or more adapters to the plurality of target polynucleotides to create a plurality of adapter-target polynucleotides, wherein the one or more adapters include a primer binding site; (b) contacting the plurality of adapter-target polynucleotides with one or more tags under conditions promoting hybridization of the one or more tags to the plurality of adapter-target polynucleotides to create a plurality of tagged adapter-target polynucleotides, wherein the one or more tags are complementary to one or more regions of the plurality of target polynucleotides; (c) contacting the plurality of tagged adapter-target polynucleotides with a solid support, under conditions promoting hybridization of the one or more tags to the solid support to create a plurality of pulldown complexes; (d) isolating the plurality of pulldown complexes; (e) optionally eluting the plurality of adapter-target polynucleotides from the plurality of pulldown complexes; (f) contacting the plurality of pulldown complexes or plurality of eluted adapter-target polynucleotides with a first polymerase under conditions promoting creation of a plurality of complementary adapter-target polynucleotides, wherein each of the complementary adapter-target polynucleotides include a 3′ protective group or a 5′ protective group; (g) contacting the plurality of adapter-target polynucleotides with a plurality of sequencing adapters in the presence of a second ligase under conditions promoting ligation of the plurality of sequencing adapters to the plurality of adapter-target polynucleotides to create a plurality of sequencing complexes; and (h) sequencing the plurality of sequencing complexes, wherein the sequencing includes sequencing of the plurality of target polynucleotide sequences without sequencing the plurality of complementary adapter-target polynucleotides, thereby sequencing the plurality of target polynucleotides, optionally wherein the plurality of target polynucleotides include one or more base modifications.

In another aspect is provided a method for detecting a plurality of base modifications in a target polynucleotide, the method including: (a) contacting the target polynucleotide with one or more adapters in the presence of a first ligase under conditions promoting ligation of the one or more adapters to the target polynucleotide to create an adapter-target polynucleotide, wherein the one or more adapters include a primer binding site; (b) contacting the adapter-target polynucleotide with one or more tags under conditions promoting hybridization of the one or more tags to the adapter-target polynucleotide to create a tagged adapter-target polynucleotide, wherein the one or more tags are complementary to one or more regions of the target polynucleotide; (c) contacting the tagged adapter-target polynucleotide with a solid support, under conditions promoting hybridization of the one or more tags to the solid support to create a pulldown complex; (d) isolating the pulldown complex; (e) optionally eluting the adapter-target polynucleotide from the pulldown complex; (f) contacting the pulldown complex or eluted adapter-target polynucleotide with a first polymerase under conditions promoting creation of a complementary adapter-target polynucleotide, wherein the complementary adapter-target polynucleotide includes a 3′ protective group or a 5′ protective group; (g) contacting the adapter-target polynucleotide with a sequencing adapter in the presence of a second ligase under conditions promoting ligation of the sequencing adapter to the adapter-target polynucleotide to create a sequencing complex; and (h) sequencing the sequencing complex, wherein the sequencing includes sequencing of the target polynucleotide sequence without sequencing the complementary adapter-target polynucleotide, thereby detecting the plurality of base modifications in the target polynucleotide.

In another aspect is provided a method for detecting a base modification in a plurality of target polynucleotides, the method including: (a) contacting the plurality of target polynucleotides with one or more adapters in the presence of a first ligase under conditions promoting ligation of the one or more adapters to the plurality of target polynucleotides to create a plurality of adapter-target polynucleotides, wherein the one or more adapters include a primer binding site; (b) contacting the plurality of adapter-target polynucleotides with one or more tags under conditions promoting hybridization of the one or more tags to the plurality of adapter-target polynucleotides to create a plurality of tagged adapter-target polynucleotides, wherein the one or more tags are complementary to one or more regions of the plurality of target polynucleotides; (c) contacting the plurality of tagged adapter-target polynucleotides with a solid support, under conditions promoting hybridization of the one or more tags to the solid support to create a plurality of pulldown complexes; (d) isolating the plurality of pulldown complexes; (e) optionally eluting the plurality of adapter-target polynucleotides from the plurality of pulldown complexes; (f) contacting the plurality of pulldown complexes or plurality of eluted adapter-target polynucleotides with a first polymerase under conditions promoting creation of a plurality of complementary adapter-target polynucleotides, wherein each of the complementary adapter-target polynucleotides include a 3′ protective group or a 5′ protective group; (g) contacting the plurality of adapter-target polynucleotides with a plurality of sequencing adapters in the presence of a second ligase under conditions promoting ligation of the plurality of sequencing adapters to the plurality of adapter-target polynucleotides to create a plurality of sequencing complexes; and (h) sequencing the plurality of sequencing complexes, wherein the sequencing includes sequencing of the plurality of target polynucleotide sequences without sequencing the plurality of complementary adapter-target polynucleotides, thereby detecting a base modifications in the plurality of target polynucleotides.

In embodiments, the conditions promoting ligation of the one or more adapters to the target polynucleotide to create an adapter-target polynucleotide include the presence of a first ligase, one or more adapters, and a target polynucleotide in an appropriate buffer and at a suitable temperature. In embodiments, the buffer includes appropriate components (e.g., pH, ionic strength, cofactors, etc.) that facilitate ligation of the one or more adapters to the target polynucleotide. In embodiments, the conditions promoting ligation of the one or more adapters to the target polynucleotide to create an adapter-target polynucleotide may include one or more adapters. In embodiments, the conditions promoting ligation of the one or more adapters to the target polynucleotide to create an adapter-target polynucleotide may include one or more barcodes. In embodiments, the conditions promoting ligation of the one or more adapters to the target polynucleotide to create an adapter-target polynucleotide may include a ligation enhancer. In embodiments, the conditions promoting ligation of the one or more adapters to the target polynucleotide to create an adapter-target polynucleotide may include a first ligase. In embodiments, the conditions promoting ligation of the one or more adapters to the target polynucleotide to create an adapter-target polynucleotide may include T4 DNA ligase. In embodiments, the conditions promoting ligation of the one or more adapters to the target polynucleotide to create an adapter-target polynucleotide may include polyethylene glycol (PEG). In embodiments, the conditions promoting ligation of the one or more adapters to the target polynucleotide to create an adapter-target polynucleotide may include adenosine triphosphate (ATP). In embodiments, the conditions promoting ligation of the one or more adapters to the target polynucleotide to create an adapter-target polynucleotide may include incubation at room temperature (RT). In embodiments, the conditions promoting ligation of the one or more adapters to the target polynucleotide to create an adapter-target polynucleotide may include incubation for about 15 minutes.

In embodiments, the conditions promoting ligation of the one or more adapters to the target polynucleotide to create an adapter-target polynucleotide may include incubation from about 16° C. to about 37° C. In embodiments, the conditions promoting ligation of the one or more adapters to the target polynucleotide to create an adapter-target polynucleotide may include incubation from about 17° C. to about 37° C. In embodiments, the conditions promoting ligation of the one or more adapters to the target polynucleotide to create an adapter-target polynucleotide may include incubation from about 18° C. to about 37° C. In embodiments, the conditions promoting ligation of the one or more adapters to the target polynucleotide to create an adapter-target polynucleotide may include incubation from about 19° C. to about 37° C. In embodiments, the conditions promoting ligation of the one or more adapters to the target polynucleotide to create an adapter-target polynucleotide may include incubation from about 20° C. to about 37° C. In embodiments, the conditions promoting ligation of the one or more adapters to the target polynucleotide to create an adapter-target polynucleotide may include incubation from about 21° C. to about 37° C. In embodiments, the conditions promoting ligation of the one or more adapters to the target polynucleotide to create an adapter-target polynucleotide may include incubation from about 22° C. to about 37° C. In embodiments, the conditions promoting ligation of the one or more adapters to the target polynucleotide to create an adapter-target polynucleotide may include incubation from about 23° C. to about 37° C. In embodiments, the conditions promoting ligation of the one or more adapters to the target polynucleotide to create an adapter-target polynucleotide may include incubation from about 24° C. to about 37° C. In embodiments, the conditions promoting ligation of the one or more adapters to the target polynucleotide to create an adapter-target polynucleotide may include incubation from about 25° C. to about 37° C. In embodiments, the conditions promoting ligation of the one or more adapters to the target polynucleotide to create an adapter-target polynucleotide may include incubation from about 26° C. to about 37° C. In embodiments, the conditions promoting ligation of the one or more adapters to the target polynucleotide to create an adapter-target polynucleotide may include incubation from about 27° C. to about 37° C. In embodiments, the conditions promoting ligation of the one or more adapters to the target polynucleotide to create an adapter-target polynucleotide may include incubation from about 28° C. to about 37° C. In embodiments, the conditions promoting ligation of the one or more adapters to the target polynucleotide to create an adapter-target polynucleotide may include incubation from about 29° C. to about 37° C. In embodiments, the conditions promoting ligation of the one or more adapters to the target polynucleotide to create an adapter-target polynucleotide may include incubation from about 30° C. to about 37° C. In embodiments, the conditions promoting ligation of the one or more adapters to the target polynucleotide to create an adapter-target polynucleotide may include incubation from about 31° C. to about 37° C. In embodiments, the conditions promoting ligation of the one or more adapters to the target polynucleotide to create an adapter-target polynucleotide may include incubation from about 32° C. to about 37° C. In embodiments, the conditions promoting ligation of the one or more adapters to the target polynucleotide to create an adapter-target polynucleotide may include incubation from about 33° C. to about 37° C. In embodiments, the conditions promoting ligation of the one or more adapters to the target polynucleotide to create an adapter-target polynucleotide may include incubation from about 34° C. to about 37° C. In embodiments, the conditions promoting ligation of the one or more adapters to the target polynucleotide to create an adapter-target polynucleotide may include incubation from about 35° C. to about 37° C. In embodiments, the conditions promoting ligation of the one or more adapters to the target polynucleotide to create an adapter-target polynucleotide may include incubation from about 36° C. to about 37° C.

In embodiments, the conditions promoting ligation of the one or more adapters to the target polynucleotide to create an adapter-target polynucleotide may include incubation from about 16° C. to about 36° C. In embodiments, the conditions promoting ligation of the one or more adapters to the target polynucleotide to create an adapter-target polynucleotide may include incubation from about 16° C. to about 35° C. In embodiments, the conditions promoting ligation of the one or more adapters to the target polynucleotide to create an adapter-target polynucleotide may include incubation from about 16° C. to about 34° C. In embodiments, the conditions promoting ligation of the one or more adapters to the target polynucleotide to create an adapter-target polynucleotide may include incubation from about 16° C. to about 33° C. In embodiments, the conditions promoting ligation of the one or more adapters to the target polynucleotide to create an adapter-target polynucleotide may include incubation from about 16° C. to about 32° C. In embodiments, the conditions promoting ligation of the one or more adapters to the target polynucleotide to create an adapter-target polynucleotide may include incubation from about 16° C. to about 31° C. In embodiments, the conditions promoting ligation of the one or more adapters to the target polynucleotide to create an adapter-target polynucleotide may include incubation from about 16° C. to about 30° C. In embodiments, the conditions promoting ligation of the one or more adapters to the target polynucleotide to create an adapter-target polynucleotide may include incubation from about 16° C. to about 29° C. In embodiments, the conditions promoting ligation of the one or more adapters to the target polynucleotide to create an adapter-target polynucleotide may include incubation from about 16° C. to about 28° C. In embodiments, the conditions promoting ligation of the one or more adapters to the target polynucleotide to create an adapter-target polynucleotide may include incubation from about 16° C. to about 27° C. In embodiments, the conditions promoting ligation of the one or more adapters to the target polynucleotide to create an adapter-target polynucleotide may include incubation from about 16° C. to about 26° C. In embodiments, the conditions promoting ligation of the one or more adapters to the target polynucleotide to create an adapter-target polynucleotide may include incubation from about 16° C. to about 25° C. In embodiments, the conditions promoting ligation of the one or more adapters to the target polynucleotide to create an adapter-target polynucleotide may include incubation from about 16° C. to about 24° C. In embodiments, the conditions promoting ligation of the one or more adapters to the target polynucleotide to create an adapter-target polynucleotide may include incubation from about 16° C. to about 23° C. In embodiments, the conditions promoting ligation of the one or more adapters to the target polynucleotide to create an adapter-target polynucleotide may include incubation from about 16° C. to about 22° C. In embodiments, the conditions promoting ligation of the one or more adapters to the target polynucleotide to create an adapter-target polynucleotide may include incubation from about 16° C. to about 21° C. In embodiments, the conditions promoting ligation of the one or more adapters to the target polynucleotide to create an adapter-target polynucleotide may include incubation from about 16° C. to about 20° C. In embodiments, the conditions promoting ligation of the one or more adapters to the target polynucleotide to create an adapter-target polynucleotide may include incubation from about 16° C. to about 19° C. In embodiments, the conditions promoting ligation of the one or more adapters to the target polynucleotide to create an adapter-target polynucleotide may include incubation from about 16° C. to about 18° C. In embodiments, the conditions promoting ligation of the one or more adapters to the target polynucleotide to create an adapter-target polynucleotide may include incubation from about 17° C. to about 36° C.

In embodiments, the conditions promoting hybridization of the one or more tags to the adapter-target polynucleotide to create a tagged adapter-target polynucleotide include the presence of one or more tags and an adapter-target polynucleotide in an appropriate buffer and at a suitable temperature. In embodiments, the buffer includes appropriate components (e.g., pH, ionic strength, cofactors, etc.) that facilitate hybridization of the one or more tags to the adapter-target polynucleotide. In embodiments, the conditions promoting hybridization of the one or more tags to the adapter-target polynucleotide to create a tagged adapter-target polynucleotide may include a hybridization enhancer. In embodiments, the conditions promoting hybridization of the one or more tags to the adapter-target polynucleotide to create a tagged adapter-target polynucleotide may include a synthetic polynucleotide. In embodiments, the synthetic polynucleotide prevents nonspecific binding. In embodiments, the conditions promoting hybridization of the one or more tags to the adapter-target polynucleotide to create a tagged adapter-target polynucleotide may include a nonspecific synthetic polynucleotide. In embodiments, the nonspecific synthetic polynucleotide prevents nonspecific binding. In embodiments, the conditions promoting hybridization of the one or more tags to the adapter-target polynucleotide to create a tagged adapter-target polynucleotide may include DNA from a different species than the target polynucleotide. In embodiments, the conditions promoting hybridization of the one or more tags to the adapter-target polynucleotide to create a tagged adapter-target polynucleotide may include salmon sperm DNA. In embodiments the salmon sperm DNA prevents nonspecific binding. In embodiments, the conditions promoting hybridization of the one or more tags to the adapter-target polynucleotide to create a tagged adapter-target polynucleotide may include incubation at about 70° C. In embodiments, the conditions promoting hybridization of the one or more tags to the adapter-target polynucleotide to create a tagged adapter-target polynucleotide may include incubation for about 20 hours.

In embodiments, the conditions promoting hybridization of the one or more tags to the adapter-target polynucleotide to create a tagged adapter-target polynucleotide may include incubation from about 50° C. to about 80° C. In embodiments, the conditions promoting hybridization of the one or more tags to the adapter-target polynucleotide to create a tagged adapter-target polynucleotide may include incubation from about 55° C. to about 80° C. In embodiments, the conditions promoting hybridization of the one or more tags to the adapter-target polynucleotide to create a tagged adapter-target polynucleotide may include incubation from about 60° C. to about 80° C. In embodiments, the conditions promoting hybridization of the one or more tags to the adapter-target polynucleotide to create a tagged adapter-target polynucleotide may include incubation from about 65° C. to about 80° C. In embodiments, the conditions promoting hybridization of the one or more tags to the adapter-target polynucleotide to create a tagged adapter-target polynucleotide may include incubation from about 70° C. to about 80° C. In embodiments, the conditions promoting hybridization of the one or more tags to the adapter-target polynucleotide to create a tagged adapter-target polynucleotide may include incubation from about 75° C. to about 80° C.

In embodiments, the conditions promoting hybridization of the one or more tags to the adapter-target polynucleotide to create a tagged adapter-target polynucleotide may include incubation from about 50° C. to about 75° C. In embodiments, the conditions promoting hybridization of the one or more tags to the adapter-target polynucleotide to create a tagged adapter-target polynucleotide may include incubation from about 50° C. to about 70° C. In embodiments, the conditions promoting hybridization of the one or more tags to the adapter-target polynucleotide to create a tagged adapter-target polynucleotide may include incubation from about 50° C. to about 65° C. In embodiments, the conditions promoting hybridization of the one or more tags to the adapter-target polynucleotide to create a tagged adapter-target polynucleotide may include incubation from about 50° C. to about 60° C. In embodiments, the conditions promoting hybridization of the one or more tags to the adapter-target polynucleotide to create a tagged adapter-target polynucleotide may include incubation from about 50° C. to about 55° C.

In embodiments, the conditions promoting hybridization of the one or more tags to the solid support to create a pulldown complex include the presence of one or more tags bound to an adapter-target polynucleotide and a solid support in an appropriate buffer and at a suitable temperature. In embodiments, the buffer includes appropriate components (e.g., pH, ionic strength, cofactors, etc.) that facilitate hybridization of the one or more tags to the solid support. In embodiments, the conditions promoting hybridization of the one or more tags to the solid support to create a pulldown complex may include incubation at about 68° C. In embodiments, the conditions promoting hybridization of the one or more tags to the solid support to create a pulldown complex may include incubation for about 5 minutes.

In embodiments, the conditions promoting creation of a complementary adapter-target polynucleotide include the presence of nucleotide triphosphates and a polymerase enzyme (e.g., a thermostable polymerase enzyme) in an appropriate buffer and at a suitable temperature. In embodiments, the buffer includes appropriate components (e.g., pH, ionic strength, cofactors, etc.) that facilitate nucleic acid synthesis. In embodiments, the conditions promoting creation of a complementary adapter-target polynucleotide may include standard polymerase chain reaction conditions. In embodiments, the conditions promoting creation of a complementary adapter-target polynucleotide may include incubation at about 60° C. In embodiments, the conditions promoting creation of a complementary adapter-target polynucleotide may include incubation for about 15 minutes. In embodiments, the conditions promoting creation of a complementary adapter-target polynucleotide may include polyethylene glycol (PEG). In embodiments, the conditions promoting creation of a complementary adapter-target polynucleotide may include adenosine triphosphate (ATP). In embodiments, the conditions promoting creation of a complementary adapter-target polynucleotide may include a Bst 3.0 DNA polymerase. In embodiments, the conditions promoting creation of a complementary adapter-target polynucleotide may include a BST buffer. In embodiments, the conditions promoting creation of a complementary adapter-target polynucleotide may include an elution primer. In embodiments, the conditions promoting creation of a complementary adapter-target polynucleotide may include one or more free nucleotides. In embodiments, the conditions promoting creation of a complementary adapter-target polynucleotide may include one or more deoxynucleotide triphosphates. In embodiments, the conditions promoting creation of a complementary adapter-target polynucleotide may include MgSO₄.

In embodiments, the conditions promoting ligation of the sequencing adapter to the adapter-target polynucleotide to create a sequencing complex include the presence of a second ligase, a sequencing adapter, and an adapter-target polynucleotide in an appropriate buffer and at a suitable temperature. In embodiments, the buffer includes appropriate components (e.g., pH, ionic strength, cofactors, etc.) that facilitate ligation of the sequencing adapter to the adapter-target polynucleotide. In embodiments, the conditions promoting ligation of the sequencing adapter to the adapter-target polynucleotide to create a sequencing complex may include incubation for about 15 minutes. In embodiments, the conditions promoting ligation of the sequencing adapter to the adapter-target polynucleotide to create a sequencing complex may include incubation at room temperature (RT). In embodiments, the conditions promoting ligation of the sequencing adapter to the adapter-target polynucleotide to create a sequencing complex may include a ligation adapter (LA). In embodiments, the conditions promoting ligation of the sequencing adapter to the adapter-target polynucleotide to create a sequencing complex may include a ligation buffer (LNB). In embodiments, the conditions promoting ligation of the sequencing adapter to the adapter-target polynucleotide to create a sequencing complex may include a Quick Ligase. In embodiments, the conditions promoting ligation of the sequencing adapter to the adapter-target polynucleotide to create a sequencing complex may include a Quick T4 Ligase.

In embodiments, the conditions promoting ligation of the sequencing adapter to the adapter-target polynucleotide to create a sequencing complex may include incubation from about 16° C. to about 37° C. In embodiments, the conditions promoting ligation of the sequencing adapter to the adapter-target polynucleotide to create a sequencing complex may include incubation from about 17° C. to about 37° C. In embodiments, the conditions promoting ligation of the sequencing adapter to the adapter-target polynucleotide to create a sequencing complex may include incubation from about 18° C. to about 37° C. In embodiments, the conditions promoting ligation of the sequencing adapter to the adapter-target polynucleotide to create a sequencing complex may include incubation from about 19° C. to about 37° C. In embodiments, the conditions promoting ligation of the sequencing adapter to the adapter-target polynucleotide to create a sequencing complex may include incubation from about 20° C. to about 37° C. In embodiments, the conditions promoting ligation of the sequencing adapter to the adapter-target polynucleotide to create a sequencing complex may include incubation from about 21° C. to about 37° C. In embodiments, the conditions promoting ligation of the sequencing adapter to the adapter-target polynucleotide to create a sequencing complex may include incubation from about 22° C. to about 37° C. In embodiments, the conditions promoting ligation of the sequencing adapter to the adapter-target polynucleotide to create a sequencing complex may include incubation from about 23° C. to about 37° C. In embodiments, the conditions promoting ligation of the sequencing adapter to the adapter-target polynucleotide to create a sequencing complex may include incubation from about 24° C. to about 37° C. In embodiments, the conditions promoting ligation of the sequencing adapter to the adapter-target polynucleotide to create a sequencing complex may include incubation from about 25° C. to about 37° C. In embodiments, the conditions promoting ligation of the sequencing adapter to the adapter-target polynucleotide to create a sequencing complex may include incubation from about 26° C. to about 37° C. In embodiments, the conditions promoting ligation of the sequencing adapter to the adapter-target polynucleotide to create a sequencing complex may include incubation from about 27° C. to about 37° C. In embodiments, the conditions promoting ligation of the sequencing adapter to the adapter-target polynucleotide to create a sequencing complex may include incubation from about 28° C. to about 37° C. In embodiments, the conditions promoting ligation of the sequencing adapter to the adapter-target polynucleotide to create a sequencing complex may include incubation from about 29° C. to about 37° C. In embodiments, the conditions promoting ligation of the sequencing adapter to the adapter-target polynucleotide to create a sequencing complex may include incubation from about 30° C. to about 37° C. In embodiments, the conditions promoting ligation of the sequencing adapter to the adapter-target polynucleotide to create a sequencing complex may include incubation from about 31° C. to about 37° C. In embodiments, the conditions promoting ligation of the sequencing adapter to the adapter-target polynucleotide to create a sequencing complex may include incubation from about 32° C. to about 37° C. In embodiments, the conditions promoting ligation of the sequencing adapter to the adapter-target polynucleotide to create a sequencing complex may include incubation from about 33° C. to about 37° C. In embodiments, the conditions promoting ligation of the sequencing adapter to the adapter-target polynucleotide to create a sequencing complex may include incubation from about 34° C. to about 37° C. In embodiments, the conditions promoting ligation of the sequencing adapter to the adapter-target polynucleotide to create a sequencing complex may include incubation from about 35° C. to about 37° C. In embodiments, the conditions promoting ligation of the sequencing adapter to the adapter-target polynucleotide to create a sequencing complex may include incubation from about 36° C. to about 37° C.

In embodiments, the conditions promoting ligation of the sequencing adapter to the adapter-target polynucleotide to create a sequencing complex may include incubation from about 16° C. to about 36° C. In embodiments, the conditions promoting ligation of the sequencing adapter to the adapter-target polynucleotide to create a sequencing complex may include incubation from about 16° C. to about 35° C. In embodiments, the conditions promoting ligation of the sequencing adapter to the adapter-target polynucleotide to create a sequencing complex may include incubation from about 16° C. to about 34° C. In embodiments, the conditions promoting ligation of the sequencing adapter to the adapter-target polynucleotide to create a sequencing complex may include incubation from about 16° C. to about 33° C. In embodiments, the conditions promoting ligation of the sequencing adapter to the adapter-target polynucleotide to create a sequencing complex may include incubation from about 16° C. to about 32° C. In embodiments, the conditions promoting ligation of the sequencing adapter to the adapter-target polynucleotide to create a sequencing complex may include incubation from about 16° C. to about 31° C. In embodiments, the conditions promoting ligation of the sequencing adapter to the adapter-target polynucleotide to create a sequencing complex may include incubation from about 16° C. to about 30° C. In embodiments, the conditions promoting ligation of the sequencing adapter to the adapter-target polynucleotide to create a sequencing complex may include incubation from about 16° C. to about 29° C. In embodiments, the conditions promoting ligation of the sequencing adapter to the adapter-target polynucleotide to create a sequencing complex may include incubation from about 16° C. to about 28° C. In embodiments, the conditions promoting ligation of the sequencing adapter to the adapter-target polynucleotide to create a sequencing complex may include incubation from about 16° C. to about 27° C. In embodiments, the conditions promoting ligation of the sequencing adapter to the adapter-target polynucleotide to create a sequencing complex may include incubation from about 16° C. to about 26° C. In embodiments, the conditions promoting ligation of the sequencing adapter to the adapter-target polynucleotide to create a sequencing complex may include incubation from about 16° C. to about 25° C. In embodiments, the conditions promoting ligation of the sequencing adapter to the adapter-target polynucleotide to create a sequencing complex may include incubation from about 16° C. to about 24° C. In embodiments, the conditions promoting ligation of the sequencing adapter to the adapter-target polynucleotide to create a sequencing complex may include incubation from about 16° C. to about 23° C. In embodiments, the conditions promoting ligation of the sequencing adapter to the adapter-target polynucleotide to create a sequencing complex may include incubation from about 16° C. to about 22° C. In embodiments, the conditions promoting ligation of the sequencing adapter to the adapter-target polynucleotide to create a sequencing complex may include incubation from about 16° C. to about 21° C. In embodiments, the conditions promoting ligation of the sequencing adapter to the adapter-target polynucleotide to create a sequencing complex may include incubation from about 16° C. to about 20° C. In embodiments, the conditions promoting ligation of the sequencing adapter to the adapter-target polynucleotide to create a sequencing complex may include incubation from about 16° C. to about 19° C. In embodiments, the conditions promoting ligation of the sequencing adapter to the adapter-target polynucleotide to create a sequencing complex may include incubation from about 16° C. to about 18° C. In embodiments, the conditions promoting ligation of the sequencing adapter to the adapter-target polynucleotide to create a sequencing complex may include incubation from about 16° C. to about 17° C.

In some embodiments, the method further includes: (i) analyzing the sequenced sequencing complex to detect the base modification.

In some embodiments, the method further includes repeating steps (a) through (i) at least once with one or more additional adapters.

In some embodiments, the target polynucleotide is isolated from a biological sample prior to step (a). In embodiments, the biological sample is from a human.

In some embodiments, the target polynucleotide includes genomic DNA (gDNA) or cell-free DNA (cfDNA). In some embodiments, the target polynucleotide includes genomic DNA (gDNA). In some embodiments, the target polynucleotide includes cell-free DNA (cfDNA).

In some embodiments, the target polynucleotide is fragmented prior to step (a). In some embodiments, the fragmenting includes mechanical fragmentation or enzymatic fragmentation. In some embodiments, the fragmenting includes mechanical fragmentation. In some embodiments, the mechanical fragmentation includes focused acoustic shearing, hydrodynamic shearing, or nebulization. In some embodiments, the mechanical fragmentation includes focused acoustic shearing. In some embodiments, the mechanical fragmentation includes hydrodynamic shearing. In some embodiments, the mechanical fragmentation includes nebulization. In some embodiments, the fragmenting includes enzymatic fragmentation. In some embodiments, the enzymatic fragmentation includes transposases, restriction enzymes, or non-specific nicking enzymes. In some embodiments, the enzymatic fragmentation includes transposases. In some embodiments, the enzymatic fragmentation includes restriction enzymes. In some embodiments, the enzymatic fragmentation includes non-specific nicking enzymes.

In some embodiments, the target polynucleotide fragments are between about 75 base pairs (bp) and about 50,000 bp. In some embodiments, the target polynucleotide fragments are between about 100 bp and about 50,000 bp. In some embodiments, the target polynucleotide fragments are between about 125 bp and about 50,000 bp. In some embodiments, the target polynucleotide fragments are between about 150 bp and about 50,000 bp. In some embodiments, the target polynucleotide fragments are between about 175 bp and about 50,000 bp. In some embodiments, the target polynucleotide fragments are between about 200 bp and about 50,000 bp. In some embodiments, the target polynucleotide fragments are between about 300 bp and about 50,000 bp. In some embodiments, the target polynucleotide fragments are between about 400 bp and about 50,000 bp. In some embodiments, the target polynucleotide fragments are between about 500 bp and about 50,000 bp. In some embodiments, the target polynucleotide fragments are between about 600 bp and about 50,000 bp. In some embodiments, the target polynucleotide fragments are between about 700 bp and about 50,000 bp. In some embodiments, the target polynucleotide fragments are between about 800 bp and about 50,000 bp. In some embodiments, the target polynucleotide fragments are between about 900 bp and about 50,000 bp. In some embodiments, the target polynucleotide fragments are between about 1000 bp and about 50,000 bp. In some embodiments, the target polynucleotide fragments are between about 2000 bp and about 50,000 bp. In some embodiments, the target polynucleotide fragments are between about 3000 bp and about 50,000 bp. In some embodiments, the target polynucleotide fragments are between about 4000 bp and about 50,000 bp. In some embodiments, the target polynucleotide fragments are between about 5000 bp and about 50,000 bp. In some embodiments, the target polynucleotide fragments are between about 6000 bp and about 50,000 bp. In some embodiments, the target polynucleotide fragments are between about 7000 bp and about 50,000 bp. In some embodiments, the target polynucleotide fragments are between about 8000 bp and about 50,000 bp. In some embodiments, the target polynucleotide fragments are between about 9000 bp and about 50,000 bp. In some embodiments, the target polynucleotide fragments are between about 10,000 bp and about 50,000 bp. In some embodiments, the target polynucleotide fragments are between about 15,000 bp and about 50,000 bp. In some embodiments, the target polynucleotide fragments are between about 20,000 bp and about 50,000 bp. In some embodiments, the target polynucleotide fragments are between about 25,000 bp and about 50,000 bp. In some embodiments, the target polynucleotide fragments are between about 30,000 bp and about 50,000 bp. In some embodiments, the target polynucleotide fragments are between about 35,000 bp and about 50,000 bp. In some embodiments, the target polynucleotide fragments are between about 40,000 bp and about 50,000 bp. In some embodiments, the target polynucleotide fragments are between about 45,000 bp and about 50,000 bp.

In some embodiments, the target polynucleotide fragments are between about 75 bp and about 45,000 bp. In some embodiments, the target polynucleotide fragments are between about 75 bp and about 40,000 bp. In some embodiments, the target polynucleotide fragments are between about 75 bp and about 35,000 bp. In some embodiments, the target polynucleotide fragments are between about 75 bp and about 30,000 bp. In some embodiments, the target polynucleotide fragments are between about 75 bp and about 25,000 bp. In some embodiments, the target polynucleotide fragments are between about 75 bp and about 20,000 bp. In some embodiments, the target polynucleotide fragments are between about 75 bp and about 15,000 bp. In some embodiments, the target polynucleotide fragments are between about 75 bp and about 10,000 bp. In some embodiments, the target polynucleotide fragments are between about 75 bp and about 9,000 bp. In some embodiments, the target polynucleotide fragments are between about 75 bp and about 8,000 bp. In some embodiments, the target polynucleotide fragments are between about 75 bp and about 7,000 bp. In some embodiments, the target polynucleotide fragments are between about 75 bp and about 6,000 bp. In some embodiments, the target polynucleotide fragments are between about 75 bp and about 5,000 bp. In some embodiments, the target polynucleotide fragments are between about 75 bp and about 4,000 bp. In some embodiments, the target polynucleotide fragments are between about 75 bp and about 3,000 bp. In some embodiments, the target polynucleotide fragments are between about 75 bp and about 2,000 bp. In some embodiments, the target polynucleotide fragments are between about 75 bp and about 1,000 bp. In some embodiments, the target polynucleotide fragments are between about 75 bp and about 900 bp. In some embodiments, the target polynucleotide fragments are between about 75 bp and about 800 bp. In some embodiments, the target polynucleotide fragments are between about 75 bp and about 700 bp. In some embodiments, the target polynucleotide fragments are between about 75 bp and about 600 bp. In some embodiments, the target polynucleotide fragments are between about 75 bp and about 500 bp. In some embodiments, the target polynucleotide fragments are between about 75 bp and about 400 bp. In some embodiments, the target polynucleotide fragments are between about 75 bp and about 300 bp. In some embodiments, the target polynucleotide fragments are between about 75 bp and about 200 bp. In some embodiments, the target polynucleotide fragments are between about 75 bp and about 175 bp. In some embodiments, the target polynucleotide fragments are between about 75 bp and about 150 bp. In some embodiments, the target polynucleotide fragments are between about 75 bp and about 125 bp. In some embodiments, the target polynucleotide fragments are between about 75 bp and about 100 bp.

In some embodiments, the target polynucleotide fragments are between 75 base bp and 50,000 bp. In some embodiments, the target polynucleotide fragments are between 100 bp and 50,000 bp. In some embodiments, the target polynucleotide fragments are between 125 bp and 50,000 bp. In some embodiments, the target polynucleotide fragments are between 150 bp and 50,000 bp. In some embodiments, the target polynucleotide fragments are between 175 bp and 50,000 bp. In some embodiments, the target polynucleotide fragments are between 200 bp and 50,000 bp. In some embodiments, the target polynucleotide fragments are between 300 bp and 50,000 bp. In some embodiments, the target polynucleotide fragments are between 400 bp and 50,000 bp. In some embodiments, the target polynucleotide fragments are between 500 bp and 50,000 bp. In some embodiments, the target polynucleotide fragments are between 600 bp and 50,000 bp. In some embodiments, the target polynucleotide fragments are between 700 bp and 50,000 bp. In some embodiments, the target polynucleotide fragments are between 800 bp and 50,000 bp. In some embodiments, the target polynucleotide fragments are between 900 bp and 50,000 bp. In some embodiments, the target polynucleotide fragments are between 1000 bp and 50,000 bp. In some embodiments, the target polynucleotide fragments are between 2000 bp and 50,000 bp. In some embodiments, the target polynucleotide fragments are between 3000 bp and 50,000 bp. In some embodiments, the target polynucleotide fragments are between 4000 bp and 50,000 bp. In some embodiments, the target polynucleotide fragments are between 5000 bp and 50,000 bp. In some embodiments, the target polynucleotide fragments are between 6000 bp and 50,000 bp. In some embodiments, the target polynucleotide fragments are between 7000 bp and 50,000 bp. In some embodiments, the target polynucleotide fragments are between 8000 bp and 50,000 bp. In some embodiments, the target polynucleotide fragments are between 9000 bp and 50,000 bp. In some embodiments, the target polynucleotide fragments are between 10,000 bp and 50,000 bp. In some embodiments, the target polynucleotide fragments are between 15,000 bp and 50,000 bp. In some embodiments, the target polynucleotide fragments are between 20,000 bp and 50,000 bp. In some embodiments, the target polynucleotide fragments are between 25,000 bp and 50,000 bp. In some embodiments, the target polynucleotide fragments are between 30,000 bp and 50,000 bp. In some embodiments, the target polynucleotide fragments are between 35,000 bp and 50,000 bp. In some embodiments, the target polynucleotide fragments are between 40,000 bp and 50,000 bp. In some embodiments, the target polynucleotide fragments are between 45,000 bp and 50,000 bp.

In some embodiments, the target polynucleotide fragments are between 75 bp and 45,000 bp. In some embodiments, the target polynucleotide fragments are between 75 bp and 40,000 bp. In some embodiments, the target polynucleotide fragments are between 75 bp and 35,000 bp. In some embodiments, the target polynucleotide fragments are between 75 bp and 30,000 bp. In some embodiments, the target polynucleotide fragments are between 75 bp and 25,000 bp. In some embodiments, the target polynucleotide fragments are between 75 bp and 20,000 bp. In some embodiments, the target polynucleotide fragments are between 75 bp and 15,000 bp. In some embodiments, the target polynucleotide fragments are between 75 bp and 10,000 bp. In some embodiments, the target polynucleotide fragments are between 75 bp and 9,000 bp. In some embodiments, the target polynucleotide fragments are between 75 bp and 8,000 bp. In some embodiments, the target polynucleotide fragments are between 75 bp and 7,000 bp. In some embodiments, the target polynucleotide fragments are between 75 bp and 6,000 bp. In some embodiments, the target polynucleotide fragments are between 75 bp and 5,000 bp. In some embodiments, the target polynucleotide fragments are between 75 bp and 4,000 bp. In some embodiments, the target polynucleotide fragments are between 75 bp and 3,000 bp. In some embodiments, the target polynucleotide fragments are between 75 bp and 2,000 bp. In some embodiments, the target polynucleotide fragments are between 75 bp and 1,000 bp. In some embodiments, the target polynucleotide fragments are between 75 bp and 900 bp. In some embodiments, the target polynucleotide fragments are between 75 bp and 800 bp. In some embodiments, the target polynucleotide fragments are between 75 bp and 700 bp. In some embodiments, the target polynucleotide fragments are between 75 bp and 600 bp. In some embodiments, the target polynucleotide fragments are between 75 bp and 500 bp. In some embodiments, the target polynucleotide fragments are between 75 bp and 400 bp. In some embodiments, the target polynucleotide fragments are between 75 bp and 300 bp. In some embodiments, the target polynucleotide fragments are between 75 bp and 200 bp. In some embodiments, the target polynucleotide fragments are between 75 bp and 175 bp. In some embodiments, the target polynucleotide fragments are between 75 bp and 150 bp. In some embodiments, the target polynucleotide fragments are between 75 bp and 125 bp. In some embodiments, the target polynucleotide fragments are between 75 bp and 100 bp.

In some embodiments, the one or more adapters further include a first barcode.

In some embodiments, the one or more additional adapters further include one or more additional barcodes.

In some embodiments, the one or more tags include one half of a cognate pair. In some embodiments, the one or more tags include one or more binding moieties. In some embodiments, the one or more tags include streptavidin, biotin, maltose, maltose binding protein, glutathione, glutathione S-transferase, chitin, chitin binding protein, an aptamer, an antigen, SpyCatcher, SpyTag, or an antibody. In some embodiments, the one or more tags include streptavidin. In some embodiments, the one or more tags include biotin. In some embodiments, the one or more tags include maltose. In some embodiments, the one or more tags include maltose binding protein. In some embodiments, the one or more tags include glutathione. In some embodiments, the one or more tags include glutathione S-transferase. In some embodiments, the one or more tags include chitin. In some embodiments, the one or more tags include chitin binding protein. In some embodiments, the one or more tags include an aptamer. In some embodiments, the one or more tags include an antigen. In some embodiments, the one or more tags include SpyCatcher. In some embodiments, the one or more tags include SpyTag. In some embodiments, the one or more tags include an antibody.

In some embodiments, the one or more tags target the same region of the target polynucleotide.

In some embodiments, the one or more tags target different regions of the target polynucleotide.

In some embodiments, the solid support includes one half of a cognate pair. In some embodiments, the solid support includes a capture moiety. In some embodiments, the solid support includes streptavidin, biotin, maltose, maltose binding protein, glutathione, glutathione S-transferase, chitin, chitin binding protein, an aptamer, an antigen, SpyCatcher, SpyTag, or an antibody. In some embodiments, the solid support includes streptavidin. In some embodiments, the solid support includes biotin. In some embodiments, the solid support includes maltose. In some embodiments, the solid support includes maltose binding protein. In some embodiments, the solid support includes glutathione. In some embodiments, the solid support includes glutathione S-transferase. In some embodiments, the solid support includes chitin. In some embodiments, the solid support includes chitin binding protein. In some embodiments, the solid support includes an aptamer. In some embodiments, the solid support includes an antigen. In some embodiments, the solid support includes SpyCatcher. In some embodiments, the solid support includes SpyTag. In some embodiments, the solid support includes an antibody.

In some embodiments, the solid support is a bead or a column. In some embodiments, the solid support is a bead. In some embodiments, the solid support is a column. In some embodiments, the bead is paramagnetic.

In some embodiments, the elution of step (e) includes separating the adapter-target polynucleotide from the one or more tags, thereby separating the adapter-targe polynucleotide from the solid support. In some embodiments, the separating of the elution of step (e) includes denaturing the one or more tags. In some embodiments, the denaturing of the one or more tags causes the one or more tags to unzip, thereby separating the adapter-target polynucleotide from the solid support. In some embodiments, the separating of the elution of step (e) includes a strand-displacing polymerase displacing the one or more tags from the adapter-target polynucleotide, thereby separating the adapter-targe polynucleotide from the solid support. In some embodiments, the separating of the elution step (e) includes a polymerase with exonuclease activity digesting the one or more tags, thereby separating the adapter-target polynucleotide from the solid support.

In some embodiments, the elution of step (e) includes NaOH, heat, a strand-displacing polymerase, or a polymerase with exonuclease activity. In some embodiments, the elution step of (e) includes a base. In embodiments, the elution of step (e) may include incubation with a base for about 5 minutes. In embodiments, the elution of step (e) may include incubation with a base at room temperature (RT). In some embodiments, the elution of step (e) includes NaOH. In embodiments, the elution of step (e) may include incubation with NaOH for about 5 minutes. In embodiments, the elution of step (e) may include incubation with NaOH at room temperature (RT). In some embodiments, the elution of step (e) includes heat. In some embodiments, the elution of step (e) includes a strand-displacing polymerase. In some embodiments, the elution of step (e) includes a polymerase with exonuclease activity.

In some embodiments, the 3′ protective group prevents sequencing of the complementary adapter-target polynucleotide. In some embodiments, the 3′ protective group includes a nucleotide overhang. In some embodiments, the nucleotide overhang includes at least one nucleotide, two nucleotides, or three nucleotides. In some embodiments, the nucleotide overhang includes at least one nucleotide. In some embodiments, the nucleotide overhang includes at least two nucleotides. In some embodiments, the nucleotide overhang includes at least three nucleotides.

In some embodiments, the 5′ protective group prevents sequencing of the complementary adapter-target polynucleotide. In some embodiments, the 5′ protective group includes an aldehyde. In some embodiments, the 5′ protective group includes an amine. In some embodiments, the 5′ protective group includes a thiol.

In some embodiments, the primer does not include a 5′ phosphate group.

In some embodiments, the sequencing adapter includes a second barcode.

In some embodiments, the base modification includes a 5-methylcytosine (5mC), a 5-hydroxymethylcytosine (5hmC), a N 6-methyladenine (6 mA), a 7-methylguanine (m7G), 2′-O-methylation (2′-O-Methyl), or a bromodeoxyuridine (BrdU). In some embodiments, the base modification includes a 5-methylcytosine (5mC). In some embodiments, the base modification includes a 5-hydroxymethylcytosine (5hmC). In some embodiments, the base modification includes a N 6-methyladenine (6 mA). In some embodiments, the base modification includes a 7-methylguanine (m7G). In some embodiments, the base modification includes 2′-O-methylation (2′-O-Methyl). In some embodiments, the base modification includes a bromodeoxyuridine (BrdU).

In some embodiments, the plurality of base modifications includes one or more of a 5-methylcytosine (5mC), a 5-hydroxymethylcytosine (5hmC), a N 6-methyladenine (6 mA), a 7-methylguanine (m7G), 2′-O-methylation (2′-O-Methyl), or a bromodeoxyuridine (BrdU). In some embodiments, the plurality of base modifications includes a 5-methylcytosine (5mC). In some embodiments, the plurality of base modifications includes a 5-hydroxymethylcytosine (5hmC). In some embodiments, the plurality of base modifications includes a N 6-methyladenine (6 mA). In some embodiments, the plurality of base modifications includes a 7-methylguanine (m7G). In some embodiments, the plurality of base modifications includes 2′-O-methylation (2′-O-Methyl). In some embodiments, the plurality of base modifications includes a bromodeoxyuridine (BrdU).

In some embodiments, the method does not include bisulfite conversion or amplification of the target polynucleotide. In some embodiments, the method does not include bisulfite conversion. In some embodiments, the method does not include amplification of the target polynucleotide.

In some embodiments, the method does not include reduced representation bisulfite sequencing (RRBS) or reduced representation methylation sequencing (RRMS). In some embodiments, the method does not include reduced representation bisulfite sequencing (RRBS). In some embodiments, the method does not include reduced representation methylation sequencing (RRMS).

In some embodiments, the first ligase or the second ligase is a T4 DNA ligase, a Quick ligase, a T3 ligase, a T7 ligase, or a Tag ligase. In some embodiments, the first ligase or the second ligase is a T4 DNA ligase. In some embodiments, the first ligase or the second ligase is a Quick ligase. In some embodiments, the first ligase or the second ligase is a T3 ligase. In some embodiments, the first ligase or the second ligase is a T7 ligase. In some embodiments, the first ligase or the second ligase is a Tag ligase.

In some embodiments, the first ligase is a T4 DNA ligase, a Quick ligase, a T3 ligase, a T7 ligase, or a Tag ligase. In some embodiments, the first ligase is a T4 DNA ligase. In some embodiments, the first ligase is a Quick ligase. In some embodiments, the first ligase is a T3 ligase. In some embodiments, the first ligase is a T7 ligase. In some embodiments, the first ligase is a Tag ligase.

In some embodiments, the second ligase is a T4 DNA ligase, a Quick ligase, a T3 ligase, a T7 ligase, or a Tag ligase. In some embodiments, the second ligase is a T4 DNA ligase. In some embodiments, the second ligase is a Quick ligase. In some embodiments, the second ligase is a T3 ligase. In some embodiments, the second ligase is a T7 ligase. In some embodiments, the second ligase is a Tag ligase.

In some embodiments, the first polymerase is a Taq polymerase, a BST polymerase, a sulfolobus polymerase, a therminator polymerase, a Klenow polymerase, a deep vent polymerase. In some embodiments, the first polymerase is a Taq polymerase. In some embodiments, the first polymerase is a BST polymerase. In some embodiments, the first polymerase is a sulfolobus polymerase. In some embodiments, the first polymerase is a therminator polymerase. In some embodiments, the first polymerase is a Klenow polymerase. In some embodiments, the first polymerase is a deep vent polymerase.

In some embodiments, the sequencing is performed on a sequencer capable of identifying modified bases.

In some embodiments, the base modification in the target polynucleotide is retained until sequencing.

In some embodiments, the method enriches the base modification in the target polynucleotide between about 300-fold to about 2400-fold compared to whole genome sequencing. In some embodiments, the method enriches the base modification in the target polynucleotide between about 400-fold to about 2400-fold compared to whole genome sequencing. In some embodiments, the method enriches the base modification in the target polynucleotide between about 500-fold to about 2400-fold compared to whole genome sequencing. In some embodiments, the method enriches the base modification in the target polynucleotide between about 600-fold to about 2400-fold compared to whole genome sequencing. In some embodiments, the method enriches the base modification in the target polynucleotide between about 700-fold to about 2400-fold compared to whole genome sequencing. In some embodiments, the method enriches the base modification in the target polynucleotide between about 800-fold to about 2400-fold compared to whole genome sequencing. In some embodiments, the method enriches the base modification in the target polynucleotide between about 900-fold to about 2400-fold compared to whole genome sequencing. In some embodiments, the method enriches the base modification in the target polynucleotide between about 1000-fold to about 2400-fold compared to whole genome sequencing. In some embodiments, the method enriches the base modification in the target polynucleotide between about 1100-fold to about 2400-fold compared to whole genome sequencing. In some embodiments, the method enriches the base modification in the target polynucleotide between about 1200-fold to about 2400-fold compared to whole genome sequencing. In some embodiments, the method enriches the base modification in the target polynucleotide between about 1300-fold to about 2400-fold compared to whole genome sequencing. In some embodiments, the method enriches the base modification in the target polynucleotide between about 1400-fold to about 2400-fold compared to whole genome sequencing. In some embodiments, the method enriches the base modification in the target polynucleotide between about 1500-fold to about 2400-fold compared to whole genome sequencing. In some embodiments, the method enriches the base modification in the target polynucleotide between about 1600-fold to about 2400-fold compared to whole genome sequencing. In some embodiments, the method enriches the base modification in the target polynucleotide between about 1700-fold to about 2400-fold compared to whole genome sequencing. In some embodiments, the method enriches the base modification in the target polynucleotide between about 1800-fold to about 2400-fold compared to whole genome sequencing. In some embodiments, the method enriches the base modification in the target polynucleotide between about 1900-fold to about 2400-fold compared to whole genome sequencing. In some embodiments, the method enriches the base modification in the target polynucleotide between about 2000-fold to about 2400-fold compared to whole genome sequencing. In some embodiments, the method enriches the base modification in the target polynucleotide between about 2100-fold to about 2400-fold compared to whole genome sequencing. In some embodiments, the method enriches the base modification in the target polynucleotide between about 2200-fold to about 2400-fold compared to whole genome sequencing. In some embodiments, the method enriches the base modification in the target polynucleotide between about 2300-fold to about 2400-fold compared to whole genome sequencing.

In some embodiments, the method enriches the base modification in the target polynucleotide between about 300-fold to about 2300-fold compared to whole genome sequencing. In some embodiments, the method enriches the base modification in the target polynucleotide between about 300-fold to about 2200-fold compared to whole genome sequencing. In some embodiments, the method enriches the base modification in the target polynucleotide between about 300-fold to about 2100-fold compared to whole genome sequencing. In some embodiments, the method enriches the base modification in the target polynucleotide between about 300-fold to about 2000-fold compared to whole genome sequencing. In some embodiments, the method enriches the base modification in the target polynucleotide between about 300-fold to about 1900-fold compared to whole genome sequencing. In some embodiments, the method enriches the base modification in the target polynucleotide between about 300-fold to about 1800-fold compared to whole genome sequencing. In some embodiments, the method enriches the base modification in the target polynucleotide between about 300-fold to about 1700-fold compared to whole genome sequencing. In some embodiments, the method enriches the base modification in the target polynucleotide between about 300-fold to about 1600-fold compared to whole genome sequencing. In some embodiments, the method enriches the base modification in the target polynucleotide between about 300-fold to about 1500-fold compared to whole genome sequencing. In some embodiments, the method enriches the base modification in the target polynucleotide between about 300-fold to about 1400-fold compared to whole genome sequencing. In some embodiments, the method enriches the base modification in the target polynucleotide between about 300-fold to about 1300-fold compared to whole genome sequencing. In some embodiments, the method enriches the base modification in the target polynucleotide between about 300-fold to about 1200-fold compared to whole genome sequencing. In some embodiments, the method enriches the base modification in the target polynucleotide between about 300-fold to about 1100-fold compared to whole genome sequencing. In some embodiments, the method enriches the base modification in the target polynucleotide between about 300-fold to about 1000-fold compared to whole genome sequencing. In some embodiments, the method enriches the base modification in the target polynucleotide between about 300-fold to about 900-fold compared to whole genome sequencing. In some embodiments, the method enriches the base modification in the target polynucleotide between about 300-fold to about 800-fold compared to whole genome sequencing. In some embodiments, the method enriches the base modification in the target polynucleotide between about 300-fold to about 700-fold compared to whole genome sequencing. In some embodiments, the method enriches the base modification in the target polynucleotide between about 300-fold to about 600-fold compared to whole genome sequencing. In some embodiments, the method enriches the base modification in the target polynucleotide between about 300-fold to about 500-fold compared to whole genome sequencing. In some embodiments, the method enriches the base modification in the target polynucleotide between about 300-fold to about 400-fold compared to whole genome sequencing.

In some embodiments, the method enriches the base modification in the target polynucleotide between 300-fold to 2400-fold compared to whole genome sequencing. In some embodiments, the method enriches the base modification in the target polynucleotide between 400-fold to 2400-fold compared to whole genome sequencing. In some embodiments, the method enriches the base modification in the target polynucleotide between 500-fold to 2400-fold compared to whole genome sequencing. In some embodiments, the method enriches the base modification in the target polynucleotide between 600-fold to 2400-fold compared to whole genome sequencing. In some embodiments, the method enriches the base modification in the target polynucleotide between 700-fold to 2400-fold compared to whole genome sequencing. In some embodiments, the method enriches the base modification in the target polynucleotide between 800-fold to 2400-fold compared to whole genome sequencing. In some embodiments, the method enriches the base modification in the target polynucleotide between 900-fold to 2400-fold compared to whole genome sequencing. In some embodiments, the method enriches the base modification in the target polynucleotide between 1000-fold to 2400-fold compared to whole genome sequencing. In some embodiments, the method enriches the base modification in the target polynucleotide between 1100-fold to 2400-fold compared to whole genome sequencing. In some embodiments, the method enriches the base modification in the target polynucleotide between 1200-fold to 2400-fold compared to whole genome sequencing. In some embodiments, the method enriches the base modification in the target polynucleotide between 1300-fold to 2400-fold compared to whole genome sequencing. In some embodiments, the method enriches the base modification in the target polynucleotide between 1400-fold to 2400-fold compared to whole genome sequencing. In some embodiments, the method enriches the base modification in the target polynucleotide between 1500-fold to 2400-fold compared to whole genome sequencing. In some embodiments, the method enriches the base modification in the target polynucleotide between 1600-fold to 2400-fold compared to whole genome sequencing. In some embodiments, the method enriches the base modification in the target polynucleotide between 1700-fold to 2400-fold compared to whole genome sequencing. In some embodiments, the method enriches the base modification in the target polynucleotide between 1800-fold to 2400-fold compared to whole genome sequencing. In some embodiments, the method enriches the base modification in the target polynucleotide between 1900-fold to 2400-fold compared to whole genome sequencing. In some embodiments, the method enriches the base modification in the target polynucleotide between 2000-fold to 2400-fold compared to whole genome sequencing. In some embodiments, the method enriches the base modification in the target polynucleotide between 2100-fold to 2400-fold compared to whole genome sequencing. In some embodiments, the method enriches the base modification in the target polynucleotide between 2200-fold to 2400-fold compared to whole genome sequencing. In some embodiments, the method enriches the base modification in the target polynucleotide between 2300-fold to 2400-fold compared to whole genome sequencing.

In some embodiments, the method enriches the base modification in the target polynucleotide between 300-fold to 2300-fold compared to whole genome sequencing. In some embodiments, the method enriches the base modification in the target polynucleotide between 300-fold to 2200-fold compared to whole genome sequencing. In some embodiments, the method enriches the base modification in the target polynucleotide between 300-fold to 2100-fold compared to whole genome sequencing. In some embodiments, the method enriches the base modification in the target polynucleotide between 300-fold to 2000-fold compared to whole genome sequencing. In some embodiments, the method enriches the base modification in the target polynucleotide between 300-fold to 1900-fold compared to whole genome sequencing. In some embodiments, the method enriches the base modification in the target polynucleotide between 300-fold to 1800-fold compared to whole genome sequencing. In some embodiments, the method enriches the base modification in the target polynucleotide between 300-fold to 1700-fold compared to whole genome sequencing. In some embodiments, the method enriches the base modification in the target polynucleotide between 300-fold to 1600-fold compared to whole genome sequencing. In some embodiments, the method enriches the base modification in the target polynucleotide between 300-fold to 1500-fold compared to whole genome sequencing. In some embodiments, the method enriches the base modification in the target polynucleotide between 300-fold to 1400-fold compared to whole genome sequencing. In some embodiments, the method enriches the base modification in the target polynucleotide between 300-fold to 1300-fold compared to whole genome sequencing. In some embodiments, the method enriches the base modification in the target polynucleotide between 300-fold to 1200-fold compared to whole genome sequencing. In some embodiments, the method enriches the base modification in the target polynucleotide between 300-fold to 1100-fold compared to whole genome sequencing. In some embodiments, the method enriches the base modification in the target polynucleotide between 300-fold to 1000-fold compared to whole genome sequencing. In some embodiments, the method enriches the base modification in the target polynucleotide between 300-fold to 900-fold compared to whole genome sequencing. In some embodiments, the method enriches the base modification in the target polynucleotide between 300-fold to 800-fold compared to whole genome sequencing. In some embodiments, the method enriches the base modification in the target polynucleotide between 300-fold to 700-fold compared to whole genome sequencing. In some embodiments, the method enriches the base modification in the target polynucleotide between 300-fold to 600-fold compared to whole genome sequencing. In some embodiments, the method enriches the base modification in the target polynucleotide between 300-fold to 500-fold compared to whole genome sequencing. In some embodiments, the method enriches the base modification in the target polynucleotide between 300-fold to 400-fold compared to whole genome sequencing.

### EXAMPLES

One skilled in the art would understand that the examples described herein are for the sole purpose of illustration, and that the present disclosure is not limited by this illustration.

### Example 1

We have created a new method to isolate specific regions of the genome using an oligo hybridization pulldown in a way that allows modified bases to be directly identified from the pulldown products. The key innovation in this method is the creation of a second strand following hybridization that is configured to only allow adapter ligation to one end. This limits sequencing to the native strand, which is the only strand with potential modifications. Thus, our process efficiently utilizes a maximum amount of sequencing reads.

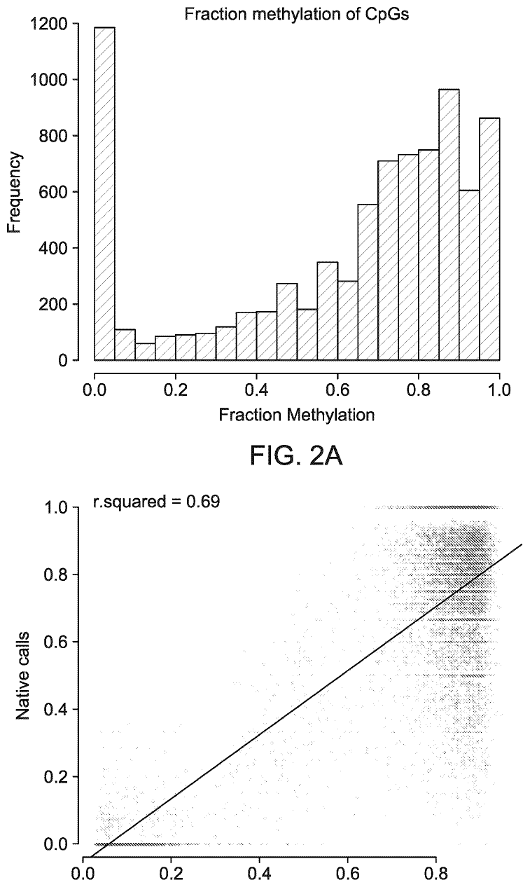

We have reduced this method to practice using a pool of polynucleotides targeting 1,235 2-kb regions of the genome whose methylation patterns can be used to assess sperm quality. The protocol used is attached. CpG loci in each region were then assessed to quantify their fraction methylation. The fraction methylation range extended from 0 (completely unmethylated) to 1 (completely methylated; FIG. 2A) and correlated with typical results using the MethylationEpic Array (FIG. 2B), a common method for identifying methylated cytosines. This finding supports our assertion that we are only sequencing the native strand. If we were sequencing both the native and synthetic strands it would be impossible to have fully methylated (fraction methylation of 1) CpGs as the synthetic strand will always be fully unmethylated. However, we had over 800 fully methylated sites. To more comprehensively confirm that we only sequenced the native strand, we also searched the sequencing reads for the reverse complement of the Nanopore Ligation Sequencing Kit (SQK-LSK114) adapter. The presence of the adapter would indicate that both ends of the enriched DNA were ligated, resulting in the sequencing of both the native and synthetic strands. Although we found the forward adapter sequence in >85,000 sequences, no reverse complement sequences were found. Together, these results show that our method sequences only the native strand, can identify a full range of base modification states, produces results consistent with other methods, and offers superior data due to the potential to achieve significantly longer reads.

### Example 2. Materials and Methods

Sample Preparation and Barcoding. Extracted DNA (1.5 ug) was fragmented, repaired, and end-prepped using the Ultra II FS DNA Module (NEB). DNA was then purified using a 0.8× ratio of Ampure XP beads (Beckman Coulter) according to the manufacturer's instructions. DNA fragments (750 ng) were mixed with 100 pmols of an adapter comprising a 3′ T overhang, a 20 basepair sequence used to identify the sample, and a 30 basepair primer binding site and ligated with Blunt/TA Mastermix (NEB) for 15 minutes at room temperature. The reaction was then stopped using 0.5 M EDTA.

Oligo Hybridization. Twelve barcoded samples were mixed and purified using a 0.5× ratio of Ampure XP beads following the manufacturer's recommendations and eluted in a 12-ul buffer containing blockers and blocker solution. In a separate tube, 20 ul of Hybridization Mix was mixed with 100 ng of the oligo probe library and heated to 95° C. for 2 min before placing on ice. The eluted DNA fragments were then heated to 95° C. for 5 min, and then both mixtures were brought to room temperature for 5 min. Both mixtures were mixed and 30 ul of Hybridization Enhancer was layered on top of the reaction. The complete mix was then incubated at 70° C. for 20 hours.

The following day, the hybridization reaction was transferred to streptavidin binding beads and incubated at 68° C. for 5 min. Following four washes, the DNA was eluted from the beads in 0.1 N NaOH. The reaction was neutralized using 4 ul of 1 M Tris, pH 5.2.

Second Strand Synthesis. Enriched DNA was mixed with 10 pmols of a primer configured to bind to the 3′ adapter and prevent ligation to that end. A second strand was synthesized using BST 3.0, which can displace any oligo probes still bound to the DNA and results in a 3′ A overhang. The reaction was incubated for 15 minutes at 60° C. to allow the second strand to be synthesized.

Library Preparation. Double-stranded, enriched DNA was used to create a nanopore sequencing library using the Ligation Sequencing Kit (nanopore) with the following modifications. First, no quantifications were conducted, as the amount of enriched DNA is below detection level. Second, the adapters were diluted 1:10 in 1×LNB buffer before ligation. Finally, up to 8 pulldowns were mixed into a single sequencing reaction.

### Example 3. Methylation-Based Predictions of Fertility Treatment Success

The example described herein is with an approach to assess the likelihood of a couple needing to utilize in vitro fertilization (IVF) to get pregnant instead of intrauterine insemination (IUI) or timed intercourse. This is based solely on targeted sequencing using the method described herein with genetic and epigenetic assessment in sperm. Our model generation produced the following results in our test group (not involved in model generation):

Predicted

| | | | Not |
|---|---|---|---|
| | | Pregnant | pregnant |
| Actual | Pregnant | 8 | 1 |
| | Not | 0 | 5 |
| | Pregnant | | |

PPV = 88.9 | NPV = 100 | Sensitivity = 100 | Specificity = 83.3

Importantly, when we tested this same model in known fertile sperm donors, 15 of 16 were predicted to achieve a pregnancy without IVF. The current model used by Inherent is built off of array data and has nowhere near the amount of information or resolution of information as produced using the methods described herein. Their results for the same 14 samples were as follows:
Predicted Poor quality (will fail without IVF)—1 of 2 individuals achieved a pregnancy without IVF.
Predicted Average quality (moderate chance of success without IVF)—4 of 8 achieved a pregnancy without IVF.
Predicted High quality (likely to get pregnancy without IVF)—1 of 4 achieved a pregnancy without IVF.

The analysis of data generated using the methods described herein dramatically improved results compared to the other tests currently on the market and the same technology can be used to improve diagnostics in many spaces.

We partnered with a company that had developed a methylation array-based method for predicting if artificial insemination will work for a particular patient. The assay looked at methylation states in sperm at CpGs in defined 2-kbp regions previously identified as informative.

We created an oligonucleotide pool to recognize the same regions and conducted Nanohyb enrichments and Nanopore Sequencing with native methylation calls. Samples consisted of DNA from 24 patients with known treatment outcomes that had previously run using the array approach. Run statistics are shown below in Table 1.

TABLE 1

| Run Statistic | Value |
|---|---|
| Number of 2,000 bp Regions (Probes) | 1,235 (4,940) |
| Percent of Genome in Region | 0.07% |
| Average Number of Reads per Sample | 183,088 |
| (filtered for quality and in region) | |
| Average Percent of Reads in Region | 57.7% |
| Average Enrichment | 824x |

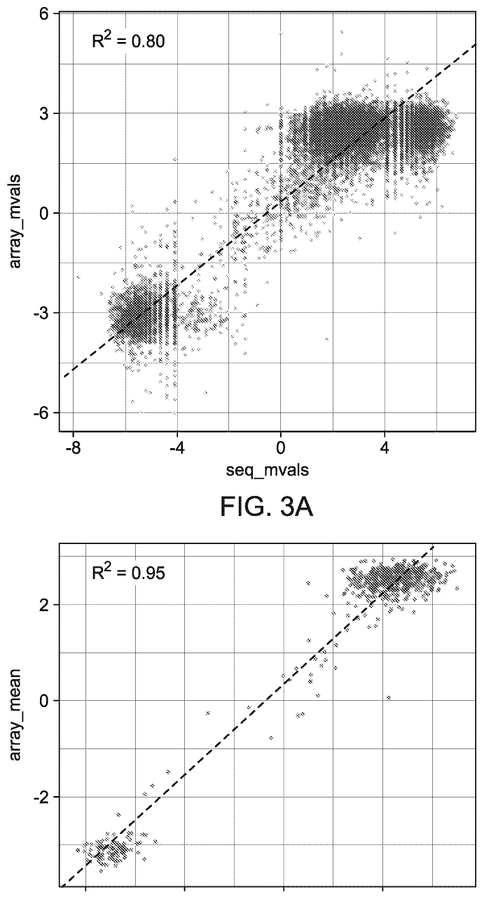

The methylation patterns were consistent with past results (FIG. 3A). In addition, the regional methylation patterns are even more consistent with the array (FIG. 3B). When average values across a 2 kb region are considered, the correlation between array and sequencing improves. However, we also found that we could gain performance by looking at methylation across an entire read, which cannot be done with an array.

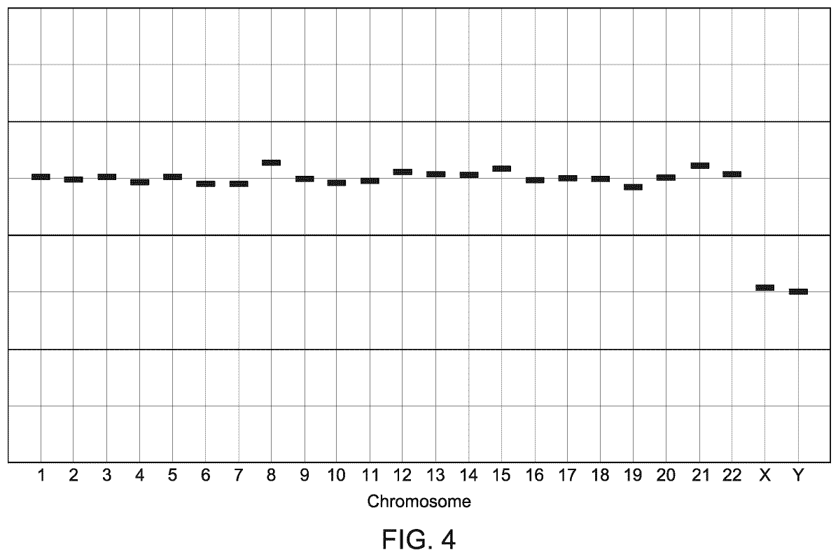

Although the probe set was not designed to measure copy number variations (CNVs), we decided to look if it could measure CNVs by measuring the average coverage of the probes along a chromosome. Chromosomal copy numbers were determined by averaging the read counts per region across the chromosome and then normalizing to the average across all autosomes. The Y chromosome had 1 probe, the X chromosome had 28 probes, and the autosomes ranged from 20 to 149 probes per chromosome. Despite the wide range of probe number, including only one on the Y chromosome, the copy number estimates were very accurate and were stable across replicates (FIG. 4).

### Example 4. Variant Detection in Hard-to-Sequence Genes

A significant portion of genes is considered “hard to sequence” due to high GC content, long lengths, repetitive regions, and the presence of similar genes or pseudogenes due to genomic duplications. One set of these genes harbor mutations that can lead to Polycystic Kidney Disease (PKD): PKD1, PKD2, and PKHD1. In addition, multiple different mutations throughout the gene have been associated with disease, and the nature of the mutation and the gene have significant effects on prognosis.

To demonstrate Nanohyb's ability to sequence these genes, we developed a probe set to these three genes and conducted a pulldown on an individual with a known mutation in PKD2. Run statistics are shown below in Table 2.

TABLE 2

| Run Statistic | Value |
|---|---|
| Number of Probes | 1,421 |
| Percent of Genome in Region | 0.015% |
| Number of Reads in Region (filtered for | 4,038 |
| quality and in region) | |
| Percent of Reads in Region | 35.4% |
| Enrichment | 2,360x |

TABLE 3
Nanohyb enrichment of long reads effectively differentiated
between duplicated genes. The pseudogenes had very few
aligned reads compared to the functional genes (bolded).

| | Number of | | Number of | | Number of |
|---|---|---|---|---|---|
| | Aligned | | Aligned | | Aligned |
| Gene | Reads | Gene | Reads | Gene | Reads |
| PKD1 | 384 | PKD2 | 547 | PKHD1 | 3,101 |
| (51.2 kb) | | (74.1 kb) | | | |
| PKD1L1 | 0 | PKD2L1 | 1 | PKHD1L1 | 0 |
| PKD1L2 | 1 | PKD2L2 | 0 | | |
| PKD1L3 | 0 | PKD2L2-DT | 0 | | |
| PKD1P1 | 3 | | | | |
| PKD1P3 | 3 | | | | |
| PKD1P4 | 2 | | | | |
| PKD1P5 | 8 | | | | |
| PKD1P6 | 5 | | | | |

### Example 5. Oligo Hybridization Enrichment for Native Strand Sequencing Exemplary Protocol

Plan 2.5 hours on the first day and 3 hours on the second day

Fragmentation and Barcoding

| | |
|---|---|
| Fragmentation (40 min) | |
| Mix the following in a PCR tube on ice (one per sample): | |
| 1.5 ug Input DNA | |
| Up to 26 ul Water | |
| 7 ul FS Buffer (Vortex first and place on ice) | |
| 2 ul FS enzyme (Vortex first and place on ice) | |
| Vortex for 5 seconds and spin down | |
| Incubate in a thermocycler at 37° C. for 3 min and then 65° C. for | |
| 30 min (hold at 4° C. after) | |
| Clean up Fragmentation Reaction (35 min) | |
| Mix the following in a 1.5 ml tube | |
| 35 ul Fragmented DNA (All) | |
| 18 ul Ampure XP Beads | |
| Incubate on Hula mixer at Room Temperature (RT) for 10 min | |
| Prepare 2 ml of fresh 80% ETOH | |
| Pellet Ampure Reaction on Magnet for 5 min | |
| Keep tube on magnet at wash with 200 ul 80% ETOH | |
| Repeat wash | |
| Remove ALL ethanol and Dry for 30 seconds to 1 min | |
| Remove from magnet as resuspend pellet in 11 ul water | |
| Incubate at 37° C. for 10 minutes, flicking every 2 min | |
| Quantify 1 ul on Qubit | |
| Dilute to 75 ng/ul | |
| Ligate Barcodes (15 min) | |
| Mix the following in a PCR tube (1 per sample): | |
| 10 ul End-Prepped DNA (All) | |
| 1 ul Barcode (100 uM) | |
| 10 ul Blunt TA MM | |
| Incubate at RT for 15 min | |
| Add 4 ul 0.5 M EDTA to stop the reaction | |
| Pool Samples and Cleanup Reaction (30 min) | |
| Mix the following in a 1.5 ml tube | Make SH Blocking Mix by |
| 300 ul DNA (25 ul × 12 samples) | diluting SH Elution |
| 150 ul Ampure XP Beads | Primer and Reverse |
| Incubate on Hula mixer at RT for 10 min | Compliment Primer to 10 |
| Thaw pulldown reagents at RT (10 uM SH Blocking Mix, Universal | uM in water |
| Blockers, Blocker Solution, Hyb mix, Probe mix) | |
| Pellet Ampure Reaction on Magnet for 5 min | |
| Keep the tube on a magnet at wash with 700 ul 80% ETOH | |
| Repeat wash | |
| Remove ALL ethanol and Dry for 30 seconds to 1 min | |
| Remove from magnet as resuspend pellet in the following: | |
| 1 ul of 10 uM SH Blocking Mix | |
| 6 ul Universal Blockers | |
| 5 ul Blocker Solution | |
| Incubate at 37° C. for 10 minutes, flicking every 2 min | |
| While incubating, place Hybridization Mix at 65° C. for 10 min | |
| Cool Hybridization Mix to RT on bench for 5 min | |
| Pellet on Magnet for 1 min and transfer supernatant to a PCR tube | |

Pulldown

Hybridize Probes (15 min + O/N)
When Hyb mix has cooled, prep Probe Solution in a PCR tube:
20 ul Hybridization Mix
100 ng of Probe Mix (library)
Water to 28 ul total
Heat Probe Solution to 95° C. for 2 min
Immediately cool it on ice for 5 min
While Probe Solution is cooling, heat Library DNA to 95° C. for 5 min
Place both the Probe Solution and Library DNA on the benchtop for
5 min
Vortex and spin the probe solution and transfer to library
Mix well by pipetting
Add 30 ul of Hybridization Enhancer to the top of the combined
reaction
Pulse spin the tube to ensure there are no bubbles
Incubate reaction at 70° C. for 20 hours in thermocycler (lid at 85° C.)
Prep Beads (15 min)
Vortex the Streptavidin Binding Beads until mixed
Add 100 ul of Streptavidin Beads to a 1.5 ml tube
Add 200 ul Binding Buffer and mix by pipetting
Place on a magnet for 1 min, remove supernatant
Resuspend beads in 200 ul Binding Buffer
Place on a magnet for 1 min, remove supernatant
Repeat wash
Repeat wash again
Add 200 ul Binding Buffer and vortex to mix beads
Heat Beads at 68° C. for 10 min in a heat block next to the
thermocycler
Preheat 225 ul of Wash Buffer 1 at 68° C.
Preheat 650 ul of Wash Buffer 2 at 48° C.
Bind Pulldown to Beads (45 minutes)
Open the thermocycler and directly transfer the hybridization
reaction (70 ul) to the Streptavidin Beads
Note: do not remove either tube from the heat
Incubate at 68° C. for 5 min, agitation is not
required
Place on magnet for 1 min, remove supernatant
Remove tube and add 200 ul of 68° C. Standard wash Buffer 1.
Mix by pipetting.
Incubate at 68° C. for 5 min
Pulse-spin to collect condensation
Transfer the entire reaction to a new 1.5 ml tube (this reduces
background from DNA binding to the tube)
Place on magnet for 1 min, remove supernatant
Remove tube and add 200 ul 48° C. Wash Buffer 2. Mix by
pipetting
Incubate at 48° C. for 5 min
Place on magnet for 1 min, remove supernatant
Repeat last four steps two more times
Use a 10 ul pipette to remove all traces of supernatant
Remove tube and add the following:
20 ul 0.1N NaOH
Incubate at RT for 5 min
Make Second Strand (30 min)
Add the following to the eluted DNA:
3 ul 10X BST buffer
1 ul 10 uM Elution Primer
1 ul dNTP
1.5 ul MgSO₄
1 ul BST 3.0
Incubate at 60° C. for 15 min
Add 20 ul of Water to bring it to 50 ul total

Library Preparation

End Prep (15 min)
Thaw the following reagents on ice:
Ultra II End Prep Buffer
Ultra Il End Prep Enzyme Mix
Ligation Adapter
Quick T4 Ligase
Thaw the following reagent at RT and place on ice:
Short Fragment Buffer (SFB)
Ligation Buffer (LNB)
Election Buffer (EB)
Combine the following in a PCR tube:
50 ul DNA
7 ul Ultra II End Prep Buffer
3 ul Ultra II End Prep Enzyme Mix
Incubate in a thermal cycler at 20° C. for 5 min and 65° C. for 5 min
(lid
at 85° C.)
Bead Cleanup (15 min)
Resuspend RT Ampure XP beads by vortexing
Add 60 ul of resuspended Ampure XP beads to the reaction
Incubate on Hula mixer at RT for 5 min
Pellet Ampure Reaction on Magnet until clear
Keep tube on magnet at wash with 700 ul 80% ETOH
Repeat wash
Remove ALL ethanol and Dry for 30 seconds to 1 min
Remove the tube from the magnetic rack and add 60 ul water
Incubate for 5 min at 37° C.
Pellet on a magnet for 1 min
Move supernatant to a new 1.5 ml tube
Adapter Ligation (20 min)
Combine the following to make Diluted Ligation Adapter:
2.5 ul Ligation Buffer (LNB)
6.5 ul Water
1 ul Ligation Adapter (LA)
Combine the following:
60 ul DNA from previous step (all)
25 ul Ligation Buffer (LNB)
10 ul Quick Ligase
5 ul Diluted Ligation Adapter
Mix by gently pipetting
Incubate the reaction for 15 min at RT
Thaw FCF and FCT at RT
Bead Cleanup (20 min)
Resuspend RT Ampure XP beads by vortexing
Add 40 ul of resuspended Ampure XP beads to the reaction
Incubate on Hula mixer at RT for 5 min
Pellet Ampure Reaction on Magnet until clear
Remove from magnet and resuspend in 250 ul Short Fragment
Buffer (SFB)
Repeat wash
Remove ALL supernatant and Dry for 30 seconds
Remove the tube from the magnetic rack and add 32 ul Elution
Buffer (EB)
Incubate for 10 min at 37° C.
Pellet on a magnet for 1 min
Move supernatant to a new 1.5 ml tube
Load Flow Cell (15 min)
Mix 30 ul of FCT into 1 vial of FCF
Vortex to mix
Load the flow cell into the P2 machine
Open the valve by turning 90 degrees clockwise
Set the P1000 to 200 ul
Insert the tip into the inlet port and turn the wheel to draw up a
small volume of yellow liquid
Load 500 ul of FCT/FCF mix
Incubate for 5 min
While incubating, mix the following in a new tube:
100 ul Sequencing Buffer (SB)
68 ul Library Beads (LIB)
32 ul DNA Library
Set up the run parameters, but do not start
Complete the priming by slowly pipetting the remaining 500 ul of
GCT/FCF mix into the inlet port
Mix the library and draw up 200 ul. Turn the dial if a space between
the end of the tip and the liquid can be seen
Place the pipette into the port and load the library
Close the valve and start the run

### Tables

TABLE 4
Nanohyb targeted locus enrichment. Two oligo pools were created
to target regions shown to have unique methylation patterns in
Sperm and Neuron cells, respectively. The percent of the genome
covered by these regions and the average percent of reads aligning
to these regions (n = 3) are shown. Fold enrichment is the
percent of reads divided by the percent of the genome targeted.

| Hybridization | % of Genome | % of Reads in | Fold Enrichment of |
|---|---|---|---|
| Pool | Targeted | Targeted Region | Targeted Regions |
| Sperm | 0.08% | 31.6% | 395.0x |
| Neuron | 0.06% | 73.0% | 1216.7x |

TABLE 5
Cost comparison for enrichment methods that allow native methylation
calling on a Nanopore. Estimates were made assuming 100,000 reads
in region per sample, 150 million reads per flow cell, and a fold
enrichment in the middle of the published ranges. Labor was estimated
at $50 per hour with 8 hours of hands on time for Nanohyb and 2 hours
for the other two methods. Price estimates were calculated using pricing
as of Sep. 13, 2023. *limited by the number of available barcodes;
the theoretical maximum is 480 samples per flow cell.

| | Fold | Samples per | Estimated Cost |
|---|---|---|---|
| Technique | Enrichment | Promethion | per Sample |
| Adaptive Sampling | 2-12x^{1,2,3} | 6 | $280.33 |
| Cas9 Enrichment | 13-54x⁴ | 24 | $176.33 |
| NanoHyb | 395-1216x | 384 | $43.63 |

### REFERENCES

1. Martin, S., Heavens, D., Lan, Y., Horsfield, S., Clark, M. D., & Leggett, R. M. (2022). Nanopore adaptive sampling: a tool for enrichment of low abundance species in metagenomic samples. Genome Biology, 23(1), 11. doi.org/10.1186/si3059-021-02582-x
2. Kovaka, S., Fan, Y., Ni, B., Timp, W., & Schatz, M. C. (2021). Targeted nanopore sequencing by real-time mapping of raw electrical signal with UNCALLED. Nature Biotechnology, 39(4), 431-441. doi.org/10.1038/s41587-020-0731-9
3. Mariya, T., Kato, T., Sugimoto, T., Miyai, S., Inagaki, H., Ohye, T., Sugihara, E., Muramatsu, Y., Mizuno, S., & Kurahashi, H. (n.d.). Target enrichment nanopore sequencing with adaptive sampling can determine the structure of the small supernumerary marker chromosomes. Nanopore Website.
4. McDonald, T. L., Zhou, W., Castro, C. P., Mumm, C., Switzenberg, J. A., Mills, R. E., & Boyle, A. P. (2021). Cas9 targeted enrichment of mobile elements using nanopore sequencing. Nature Communications, 12(1), 3586. doi.org/10.1038/s41467-021-23918-y

### EMBODIMENTS

Embodiment 1. A method for detecting a base modification in a target polynucleotide, the method comprising: (a) contacting the target polynucleotide with one or more adapters in the presence of a first ligase under conditions promoting ligation of the one or more adapters to the target polynucleotide to create an adapter-target polynucleotide, wherein the one or more adapters comprise a primer binding site; (b) contacting the adapter-target polynucleotide with one or more tags under conditions promoting hybridization of the one or more tags to the adapter-target polynucleotide to create a tagged adapter-target polynucleotide, wherein the one or more tags are complementary to one or more regions of the target polynucleotide; (c) contacting the tagged adapter-target polynucleotide with a solid support, under conditions promoting hybridization of the one or more tags to the solid support to create a pulldown complex; (d) isolating the pulldown complex; (e) optionally eluting the adapter-target polynucleotide from the pulldown complex; (f) contacting the pulldown complex or eluted adapter-target polynucleotide with a first polymerase under conditions promoting creation of a complementary adapter-target polynucleotide, wherein the complementary adapter-target polynucleotide comprises a 3′ protective group; (g) contacting the adapter-target polynucleotide with a sequencing adapter in the presence of a second ligase under conditions promoting ligation of the sequencing adapter to the adapter-target polynucleotide to create a sequencing complex; and (h) sequencing the sequencing complex, wherein the sequencing comprises sequencing of the target polynucleotide sequence without sequencing the complementary adapter-target polynucleotide thereby detecting the base modification in the target polynucleotide.
Embodiment 2. A method for sequencing a target polynucleotide, the method comprising: (a) contacting the target polynucleotide with one or more adapters in the presence of a first ligase under conditions promoting ligation of the one or more adapters to the target polynucleotide to create an adapter-target polynucleotide, wherein the one or more adapters comprise a primer binding site; (b) contacting the adapter-target polynucleotide with one or more tags under conditions promoting hybridization of the one or more tags to the adapter-target polynucleotide to create a tagged adapter-target polynucleotide, wherein the one or more tags are complementary to one or more regions of the target polynucleotide; (c) contacting the tagged adapter-target polynucleotide with a solid support, under conditions promoting hybridization of the one or more tags to the solid support to create a pulldown complex; (d) isolating the pulldown complex; (e) optionally eluting the adapter-target polynucleotide from the pulldown complex; (f) contacting the pulldown complex or eluted adapter-target polynucleotide with a first polymerase under conditions promoting creation of a complementary adapter-target polynucleotide, wherein the complementary adapter-target polynucleotide comprises a 3′ protective group; (g) contacting the adapter-target polynucleotide with a sequencing adapter in the presence of a second ligase under conditions promoting ligation of the sequencing adapter to the adapter-target polynucleotide to create a sequencing complex; and (h) sequencing the sequencing complex, wherein the sequencing comprises sequencing of the target polynucleotide sequence without sequencing the complementary adapter-target polynucleotide, thereby sequencing the target polynucleotide, optionally wherein the target polynucleotide comprises one or more base modifications.
Embodiment 3. A method for quantifying a base modification in a target polynucleotide, the method comprising: (a) contacting the target polynucleotide with one or more adapters in the presence of a first ligase under conditions promoting ligation of the one or more adapters to the target polynucleotide to create an adapter-target polynucleotide, wherein the one or more adapters comprise a primer binding site; (b) contacting the adapter-target polynucleotide with one or more tags under conditions promoting hybridization of the one or more tags to the adapter-target polynucleotide or the adapter-target polynucleotide to the one or more tags to create a tagged adapter-target polynucleotide, wherein the one or more tags are complementary to one or more regions of the target polynucleotide; (c) contacting the tagged adapter-target polynucleotide with a solid support, under conditions promoting hybridization of the one or more tags to the solid support to create a pulldown complex; (d) isolating the pulldown complex; (e) optionally eluting the adapter-target polynucleotide from the pulldown complex; (f) contacting the pulldown complex or eluted adapter-target polynucleotide with a first polymerase under conditions promoting creation of a complementary adapter-target polynucleotide, wherein the complementary adapter-target polynucleotide comprises a 3′ protective group; (g) contacting the adapter-target polynucleotide with a sequencing adapter in the presence of a second ligase under conditions promoting ligation of the sequencing adapter to the adapter-target polynucleotide to create a sequencing complex; and (h) sequencing the sequencing complex, wherein the sequencing comprises sequencing of the target polynucleotide sequence without sequencing the complementary adapter-target polynucleotide, thereby quantifying the base modification in the target polynucleotide.
Embodiment 4. A method for detecting a plurality of base modifications in a plurality of target polynucleotides, the method comprising: (a) contacting the plurality of target polynucleotides with one or more adapters in the presence of a first ligase under conditions promoting ligation of the one or more adapters to the plurality of target polynucleotides to create a plurality of adapter-target polynucleotides, wherein the one or more adapters comprise a primer binding site; (b) contacting the plurality of adapter-target polynucleotides with one or more tags under conditions promoting hybridization of the one or more tags to the plurality of adapter-target polynucleotides to create a plurality of tagged adapter-target polynucleotides, wherein the one or more tags are complementary to one or more regions of the plurality of target polynucleotides; (c) contacting the plurality of tagged adapter-target polynucleotides with a solid support, under conditions promoting hybridization of the one or more tags to the solid support to create a plurality of pulldown complexes; (d) isolating the plurality of pulldown complexes; (e) optionally eluting the plurality of adapter-target polynucleotides from the plurality of pulldown complexes; (f) contacting the plurality of pulldown complexes or plurality of eluted adapter-target polynucleotides with a first polymerase under conditions promoting creation of a plurality of complementary adapter-target polynucleotides, wherein each of the complementary adapter-target polynucleotides comprise a 3′ protective group; (g) contacting the plurality of adapter-target polynucleotides with a plurality of sequencing adapters in the presence of a second ligase under conditions promoting ligation of the plurality of sequencing adapters to the plurality of adapter-target polynucleotides to create a plurality of sequencing complexes; and (h) sequencing the plurality of sequencing complexes, wherein the sequencing comprises sequencing of the plurality of target polynucleotide sequences without sequencing the plurality of complementary adapter-target polynucleotides, thereby detecting a plurality of base modifications in the plurality of target polynucleotides.
Embodiment 5. A method for sequencing a plurality of target polynucleotides, the method comprising: (a) contacting the plurality of target polynucleotides with one or more adapters in the presence of a first ligase under conditions promoting ligation of the one or more adapters to the plurality of target polynucleotides to create a plurality of adapter-target polynucleotides, wherein the one or more adapters comprise a primer binding site; (b) contacting the plurality of adapter-target polynucleotides with one or more tags under conditions promoting hybridization of the one or more tags to the plurality of adapter-target polynucleotides to create a plurality of tagged adapter-target polynucleotides, wherein the one or more tags are complementary to one or more regions of the plurality of target polynucleotides; (c) contacting the plurality of tagged adapter-target polynucleotides with a solid support, under conditions promoting hybridization of the one or more tags to the solid support to create a plurality of pulldown complexes; (d) isolating the plurality of pulldown complexes; (e) optionally eluting the plurality of adapter-target polynucleotides from the plurality of pulldown complexes; (f) contacting the plurality of pulldown complexes or plurality of eluted adapter-target polynucleotides with a first polymerase under conditions promoting creation of a plurality of complementary adapter-target polynucleotides, wherein each of the complementary adapter-target polynucleotides comprise a 3′ protective group; (g) contacting the plurality of adapter-target polynucleotides with a plurality of sequencing adapters in the presence of a second ligase under conditions promoting ligation of the plurality of sequencing adapters to the plurality of adapter-target polynucleotides to create a plurality of sequencing complexes; and (h) sequencing the plurality of sequencing complexes, wherein the sequencing comprises sequencing of the plurality of target polynucleotide sequences without sequencing the plurality of complementary adapter-target polynucleotides, thereby sequencing the plurality of target polynucleotides, optionally wherein the plurality of target polynucleotides comprise one or more base modifications.
Embodiment 6. A method for detecting a plurality of base modifications in a target polynucleotide, the method comprising: (a) contacting the target polynucleotide with one or more adapters in the presence of a first ligase under conditions promoting ligation of the one or more adapters to the target polynucleotide to create an adapter-target polynucleotide, wherein the one or more adapters comprise a primer binding site; (b) contacting the adapter-target polynucleotide with one or more tags under conditions promoting hybridization of the one or more tags to the adapter-target polynucleotide to create a tagged adapter-target polynucleotide, wherein the one or more tags are complementary to one or more regions of the target polynucleotide; (c) contacting the tagged adapter-target polynucleotide with a solid support, under conditions promoting hybridization of the one or more tags to the solid support to create a pulldown complex; (d) isolating the pulldown complex; (e) optionally eluting the adapter-target polynucleotide from the pulldown complex; (f) contacting the pulldown complex or eluted adapter-target polynucleotide with a first polymerase under conditions promoting creation of a complementary adapter-target polynucleotide, wherein the complementary adapter-target polynucleotide comprises a 3′ protective group; (g) contacting the adapter-target polynucleotide with a sequencing adapter in the presence of a second ligase under conditions promoting ligation of the sequencing adapter to the adapter-target polynucleotide to create a sequencing complex; and (h) sequencing the sequencing complex, wherein the sequencing comprises sequencing of the target polynucleotide sequence without sequencing the complementary adapter-target polynucleotide, thereby detecting the plurality of base modifications in the target polynucleotide.
Embodiment 7. A method for detecting a base modification in a plurality of target polynucleotides, the method comprising: (a) contacting the plurality of target polynucleotides with one or more adapters in the presence of a first ligase under conditions promoting ligation of the one or more adapters to the plurality of target polynucleotides to create a plurality of adapter-target polynucleotides, wherein the one or more adapters comprise a primer binding site; (b) contacting the plurality of adapter-target polynucleotides with one or more tags under conditions promoting hybridization of the one or more tags to the plurality of adapter-target polynucleotides to create a plurality of tagged adapter-target polynucleotides, wherein the one or more tags are complementary to one or more regions of the plurality of target polynucleotides; (c) contacting the plurality of tagged adapter-target polynucleotides with a solid support, under conditions promoting hybridization of the one or more tags to the solid support to create a plurality of pulldown complexes; (d) isolating the plurality of pulldown complexes; (e) optionally eluting the plurality of adapter-target polynucleotides from the plurality of pulldown complexes; (f) contacting the plurality of pulldown complexes or plurality of eluted adapter-target polynucleotides with a first polymerase under conditions promoting creation of a plurality of complementary adapter-target polynucleotides, wherein each of the complementary adapter-target polynucleotides comprise a 3′ protective group; (g) contacting the plurality of adapter-target polynucleotides with a plurality of sequencing adapters in the presence of a second ligase under conditions promoting ligation of the plurality of sequencing adapters to the plurality of adapter-target polynucleotides to create a plurality of sequencing complexes; and (h) sequencing the plurality of sequencing complexes, wherein the sequencing comprises sequencing of the plurality of target polynucleotide sequences without sequencing the plurality of complementary adapter-target polynucleotides, thereby detecting a base modifications in the plurality of target polynucleotides.
Embodiment 8. The method of any one of the above embodiments, further comprising: (i) analyzing the sequenced sequencing complex to detect the base modification.
Embodiment 9. The method of any one of the above embodiments, wherein the sequencing comprises identifying abase modification in the target polynucleotide.
Embodiment 10. The method of any one of the above embodiments, further comprising repeating steps (a) through (i) at least once with one or more additional adapters.
Embodiment 11. The method of any one of the above embodiments, wherein the target polynucleotide is isolated from a biological sample prior to step (a).
Embodiment 12. The method of embodiment 11, wherein the biological sample is from a human.
Embodiment 13. The method of any one of the above embodiments, wherein the target polynucleotide comprises genomic DNA (gDNA) or cell-free DNA (cfDNA).
Embodiment 14. The method of any one of the above embodiments, wherein the target polynucleotide is fragmented prior to step (a).
Embodiment 15. The method of any one of the above embodiments, wherein the one or more adapters further comprise a first barcode.
Embodiment 16. The method of embodiment 15, wherein the one or more additional adapters further comprise one or more additional barcodes.
Embodiment 17. The method of any one of the above embodiments, wherein the one or more tags comprise streptavidin, biotin, maltose, maltose binding protein, glutathione, glutathione S-transferase, chitin, chitin binding protein, an aptamer, an antigen, SpyCatcher, SpyTag, or an antibody.
Embodiment 18. The method of any one of the above embodiments, wherein the one or more tags target the same region of the target polynucleotide.
Embodiment 19. The method of any one of embodiments 1-17, wherein the one or more tags target different regions of the target polynucleotide.
Embodiment 20. The method of any one of the above embodiments, wherein the solid support comprises streptavidin, biotin, maltose, maltose binding protein, glutathione, glutathione S-transferase, chitin, chitin binding protein, an aptamer, an antigen, SpyCatcher, SpyTag, or an antibody.
Embodiment 21. The method of any one of the above embodiments, wherein the solid support is a bead or a column.
Embodiment 22. The method of embodiment 21, wherein the bead is paramagnetic.
Embodiment 23. The method of any one of the above embodiments, wherein the elution of step (e) comprises NaOH, heat, a strand-displacing polymerase, or a polymerase with exonuclease activity.
Embodiment 24. The method of any one of the above embodiments, wherein the 3′ protective group prevents sequencing of the complementary adapter-target polynucleotide.
Embodiment 25. The method of any one of the above embodiments, wherein the 3′ protective group comprises a nucleotide overhang.
Embodiment 26. The method of embodiment 25, wherein the nucleotide overhang comprises at least one nucleotide, two nucleotides, or three nucleotides.
Embodiment 27. The method of any one of the above embodiments, the primer does not comprise a 5′ phosphate group.
Embodiment 28. The method of any one of the above embodiments, the sequencing adapter comprises a second barcode.
Embodiment 29. The method of any one of the above embodiments, wherein the base modification comprises a 5-methylcytosine (5mC), a 5-hydroxymethylcytosine (5hmC), a N 6-methyladenine (6 mA), a 7-methylguanine (m7G), 2′-O-methylation (2′-O-Methyl), or a bromodeoxyuridine (BrdU).
Embodiment 30. The method of any one of the above embodiments, wherein the method does not comprise bisulfite conversion or amplification of the target polynucleotide.
Embodiment 31. The method of any one of the above embodiments, wherein the method does not comprise reduced representation bisulfite sequencing (RRBS) or reduced representation methylation sequencing (RRMS).
Embodiment 32. The method of any one of the above embodiments, wherein the first ligase or the second ligase is a T4 DNA ligase, a Quick ligase, a T3 ligase, a T7 ligase, or a Tag ligase.
Embodiment 33. The method of any one of the above embodiments, wherein the first polymerase is a Taq polymerase, a BST polymerase, a sulfolobus polymerase, a therminator polymerase, a Klenow polymerase, a deep vent polymerase.
Embodiment 34. The method of any one of the above embodiments, wherein sequencing is performed on a sequencer capable of identifying modified bases.
Embodiment 35. The method of any one of the above embodiments, wherein the base modification in the target polynucleotide is retained until sequencing.
Embodiment 36. The method of any one of the above embodiments, wherein the method enriches the base modification in the target polynucleotide between about 300-fold to about 2400-fold compared to whole genome sequencing.

What is claimed is:

1. A method for detecting a base modification in a target polynucleotide, the method comprising:
(a) contacting the target polynucleotide with one or more adapters in the presence of a first ligase under conditions promoting ligation of the one or more adapters to the target polynucleotide to create an adapter-target polynucleotide, wherein the one or more adapters comprise a primer binding site;
(b) contacting the adapter-target polynucleotide with one or more tags under conditions promoting hybridization of the one or more tags to the adapter-target polynucleotide to create a tagged adapter-target polynucleotide, wherein the one or more tags are complementary to one or more regions of the target polynucleotide;
(c) contacting the tagged adapter-target polynucleotide with a solid support, under conditions promoting hybridization of the one or more tags to the solid support to create a pulldown complex;
(d) isolating the pulldown complex;
(e) optionally eluting the adapter-target polynucleotide from the pulldown complex;
(f) contacting the pulldown complex or eluted adapter-target polynucleotide with a first polymerase under conditions promoting creation of a complementary adapter-target polynucleotide, wherein the complementary adapter-target polynucleotide comprises a 3′ protective group or a 5′ protective group;
(g) contacting the adapter-target polynucleotide with a sequencing adapter in the presence of a second ligase under conditions promoting ligation of the sequencing adapter to the adapter-target polynucleotide to create a sequencing complex; and
(h) sequencing the sequencing complex, wherein the sequencing comprises sequencing of the target polynucleotide sequence without sequencing the complementary adapter-target polynucleotide,
thereby detecting the base modification in the target polynucleotide.

2. A method for sequencing a target polynucleotide, the method comprising:
(a) contacting the target polynucleotide with one or more adapters in the presence of a first ligase under conditions promoting ligation of the one or more adapters to the target polynucleotide to create an adapter-target polynucleotide, wherein the one or more adapters comprise a primer binding site;
(b) contacting the adapter-target polynucleotide with one or more tags under conditions promoting hybridization of the one or more tags to the adapter-target polynucleotide to create a tagged adapter-target polynucleotide, wherein the one or more tags are complementary to one or more regions of the target polynucleotide;
(c) contacting the tagged adapter-target polynucleotide with a solid support, under conditions promoting hybridization of the one or more tags to the solid support to create a pulldown complex;
(d) isolating the pulldown complex;
(e) optionally eluting the adapter-target polynucleotide from the pulldown complex;
(f) contacting the pulldown complex or eluted adapter-target polynucleotide with a first polymerase under conditions promoting creation of a complementary adapter-target polynucleotide, wherein the complementary adapter-target polynucleotide comprises a 3′ protective group or a 5′ protective group;
(g) contacting the adapter-target polynucleotide with a sequencing adapter in the presence of a second ligase under conditions promoting ligation of the sequencing adapter to the adapter-target polynucleotide to create a sequencing complex; and
(h) sequencing the sequencing complex, wherein the sequencing comprises sequencing of the target polynucleotide sequence without sequencing the complementary adapter-target polynucleotide,
thereby sequencing the target polynucleotide, optionally wherein the target polynucleotide comprises one or more base modifications.

3. A method for quantifying a base modification in a target polynucleotide, the method comprising:
(a) contacting the target polynucleotide with one or more adapters in the presence of a first ligase under conditions promoting ligation of the one or more adapters to the target polynucleotide to create an adapter-target polynucleotide, wherein the one or more adapters comprise a primer binding site;
(b) contacting the adapter-target polynucleotide with one or more tags under conditions promoting hybridization of the one or more tags to the adapter-target polynucleotide or the adapter-target polynucleotide to the one or more tags to create a tagged adapter-target polynucleotide, wherein the one or more tags are complementary to one or more regions of the target polynucleotide;
(c) contacting the tagged adapter-target polynucleotide with a solid support, under conditions promoting hybridization of the one or more tags to the solid support to create a pulldown complex;
(d) isolating the pulldown complex;
(e) optionally eluting the adapter-target polynucleotide from the pulldown complex;
(f) contacting the pulldown complex or eluted adapter-target polynucleotide with a first polymerase under conditions promoting creation of a complementary adapter-target polynucleotide, wherein the complementary adapter-target polynucleotide comprises a 3′ protective group or a 5′ protective group;
(g) contacting the adapter-target polynucleotide with a sequencing adapter in the presence of a second ligase under conditions promoting ligation of the sequencing adapter to the adapter-target polynucleotide to create a sequencing complex; and
(h) sequencing the sequencing complex, wherein the sequencing comprises sequencing of the target polynucleotide sequence without sequencing the complementary adapter-target polynucleotide,
thereby quantifying the base modification in the target polynucleotide.

4. A method for detecting a plurality of base modifications in a plurality of target polynucleotides, the method comprising:
(a) contacting the plurality of target polynucleotides with one or more adapters in the presence of a first ligase under conditions promoting ligation of the one or more adapters to the plurality of target polynucleotides to create a plurality of adapter-target polynucleotides, wherein the one or more adapters comprise a primer binding site;
(b) contacting the plurality of adapter-target polynucleotides with one or more tags under conditions promoting hybridization of the one or more tags to the plurality of adapter-target polynucleotides to create a plurality of tagged adapter-target polynucleotides, wherein the one or more tags are complementary to one or more regions of the plurality of target polynucleotides;
(c) contacting the plurality of tagged adapter-target polynucleotides with a solid support, under conditions promoting hybridization of the one or more tags to the solid support to create a plurality of pulldown complexes;
(d) isolating the plurality of pulldown complexes;
(e) optionally eluting the plurality of adapter-target polynucleotides from the plurality of pulldown complexes;
(f) contacting the plurality of pulldown complexes or plurality of eluted adapter-target polynucleotides with a first polymerase under conditions promoting creation of a plurality of complementary adapter-target polynucleotides, wherein each of the complementary adapter-target polynucleotides comprise a 3′ protective group or a 5′ protective group;
(g) contacting the plurality of adapter-target polynucleotides with a plurality of sequencing adapters in the presence of a second ligase under conditions promoting ligation of the plurality of sequencing adapters to the plurality of adapter-target polynucleotides to create a plurality of sequencing complexes; and
(h) sequencing the plurality of sequencing complexes, wherein the sequencing comprises sequencing of the plurality of target polynucleotide sequences without sequencing the plurality of complementary adapter-target polynucleotides,
thereby detecting a plurality of base modifications in the plurality of target polynucleotides.

5. A method for sequencing a plurality of target polynucleotides, the method comprising:
(a) contacting the plurality of target polynucleotides with one or more adapters in the presence of a first ligase under conditions promoting ligation of the one or more adapters to the plurality of target polynucleotides to create a plurality of adapter-target polynucleotides, wherein the one or more adapters comprise a primer binding site;
(b) contacting the plurality of adapter-target polynucleotides with one or more tags under conditions promoting hybridization of the one or more tags to the plurality of adapter-target polynucleotides to create a plurality of tagged adapter-target polynucleotides, wherein the one or more tags are complementary to one or more regions of the plurality of target polynucleotides;
(c) contacting the plurality of tagged adapter-target polynucleotides with a solid support, under conditions promoting hybridization of the one or more tags to the solid support to create a plurality of pulldown complexes;
(d) isolating the plurality of pulldown complexes;
(e) optionally eluting the plurality of adapter-target polynucleotides from the plurality of pulldown complexes;
(f) contacting the plurality of pulldown complexes or plurality of eluted adapter-target polynucleotides with a first polymerase under conditions promoting creation of a plurality of complementary adapter-target polynucleotides, wherein each of the complementary adapter-target polynucleotides comprise a 3′ protective group or a 5′ protective group;
(g) contacting the plurality of adapter-target polynucleotides with a plurality of sequencing adapters in the presence of a second ligase under conditions promoting ligation of the plurality of sequencing adapters to the plurality of adapter-target polynucleotides to create a plurality of sequencing complexes; and
(h) sequencing the plurality of sequencing complexes, wherein the sequencing comprises sequencing of the plurality of target polynucleotide sequences without sequencing the plurality of complementary adapter-target polynucleotides,
thereby sequencing the plurality of target polynucleotides, optionally wherein the plurality of target polynucleotides comprise one or more base modifications.

6. A method for detecting a plurality of base modifications in a target polynucleotide, the method comprising:
(a) contacting the target polynucleotide with one or more adapters in the presence of a first ligase under conditions promoting ligation of the one or more adapters to the target polynucleotide to create an adapter-target polynucleotide, wherein the one or more adapters comprise a primer binding site;
(b) contacting the adapter-target polynucleotide with one or more tags under conditions promoting hybridization of the one or more tags to the adapter-target polynucleotide to create a tagged adapter-target polynucleotide, wherein the one or more tags are complementary to one or more regions of the target polynucleotide;
(c) contacting the tagged adapter-target polynucleotide with a solid support, under conditions promoting hybridization of the one or more tags to the solid support to create a pulldown complex;
(d) isolating the pulldown complex;
(e) optionally eluting the adapter-target polynucleotide from the pulldown complex;
(f) contacting the pulldown complex or eluted adapter-target polynucleotide with a first polymerase under conditions promoting creation of a complementary adapter-target polynucleotide, wherein the complementary adapter-target polynucleotide comprises a 3′ protective group or a 5′ protective group;
(g) contacting the adapter-target polynucleotide with a sequencing adapter in the presence of a second ligase under conditions promoting ligation of the sequencing adapter to the adapter-target polynucleotide to create a sequencing complex; and
(h) sequencing the sequencing complex, wherein the sequencing comprises sequencing of the target polynucleotide sequence without sequencing the complementary adapter-target polynucleotide,
thereby detecting the plurality of base modifications in the target polynucleotide.

7. A method for detecting a base modification in a plurality of target polynucleotides, the method comprising:
(a) contacting the plurality of target polynucleotides with one or more adapters in the presence of a first ligase under conditions promoting ligation of the one or more adapters to the plurality of target polynucleotides to create a plurality of adapter-target polynucleotides, wherein the one or more adapters comprise a primer binding site;
(b) contacting the plurality of adapter-target polynucleotides with one or more tags under conditions promoting hybridization of the one or more tags to the plurality of adapter-target polynucleotides to create a plurality of tagged adapter-target polynucleotides, wherein the one or more tags are complementary to one or more regions of the plurality of target polynucleotides;
(c) contacting the plurality of tagged adapter-target polynucleotides with a solid support, under conditions promoting hybridization of the one or more tags to the solid support to create a plurality of pulldown complexes;
(d) isolating the plurality of pulldown complexes;
(e) optionally eluting the plurality of adapter-target polynucleotides from the plurality of pulldown complexes;
(f) contacting the plurality of pulldown complexes or plurality of eluted adapter-target polynucleotides with a first polymerase under conditions promoting creation of a plurality of complementary adapter-target polynucleotides, wherein each of the complementary adapter-target polynucleotides comprise a 3′ protective group or a 5′ protective group;
(g) contacting the plurality of adapter-target polynucleotides with a plurality of sequencing adapters in the presence of a second ligase under conditions promoting ligation of the plurality of sequencing adapters to the plurality of adapter-target polynucleotides to create a plurality of sequencing complexes; and
(h) sequencing the plurality of sequencing complexes, wherein the sequencing comprises sequencing of the plurality of target polynucleotide sequences without sequencing the plurality of complementary adapter-target polynucleotides,
thereby detecting a base modifications in the plurality of target polynucleotides.

8. The method of any one of the above claims, further comprising:
(i) analyzing the sequenced sequencing complex to detect the base modification.

9. The method of any one of the above claims, wherein the sequencing comprises identifying a base modification in the target polynucleotide.

10. The method of any one of the above claims, further comprising repeating steps (a) through (i) at least once with one or more additional adapters.

11. The method of any one of the above claims, wherein the target polynucleotide is isolated from a biological sample prior to step (a).

12. The method of claim 11, wherein the biological sample is from a human.

13. The method of any one of the above claims, wherein the target polynucleotide comprises genomic DNA (gDNA) or cell-free DNA (cfDNA).

14. The method of any one of the above claims, wherein the target polynucleotide is fragmented prior to step (a).

15. The method of any one of the above claims, wherein the one or more adapters further comprise a first barcode.

16. The method of claim 15, wherein the one or more additional adapters further comprise one or more additional barcodes.

17. The method of any one of the above claims, wherein the one or more tags comprise streptavidin, biotin, maltose, maltose binding protein, glutathione, glutathione S-transferase, chitin, chitin binding protein, an aptamer, an antigen, SpyCatcher, SpyTag, or an antibody.

18. The method of any one of the above claims, wherein the one or more tags target the same region of the target polynucleotide.

19. The method of any one of claims 1-17, wherein the one or more tags target different regions of the target polynucleotide.

20. The method of any one of the above claims, wherein the solid support comprises streptavidin, biotin, maltose, maltose binding protein, glutathione, glutathione S-transferase, chitin, chitin binding protein, an aptamer, an antigen, SpyCatcher, SpyTag, or an antibody.

21. The method of any one of the above claims, wherein the solid support is a bead or a column.

22. The method of claim 21, wherein the bead is paramagnetic.

23. The method of any one of the above claims, wherein the elution of step (e) comprises NaOH, heat, a strand-displacing polymerase, or a polymerase with exonuclease activity.

24. The method of any one of the above claims, wherein the 3′ protective group or the 5′ protective group prevents sequencing of the complementary adapter-target polynucleotide.

25. The method of any one of the above claims, wherein the 3′ protective group comprises a nucleotide overhang.

26. The method of claim 25, wherein the nucleotide overhang comprises at least one nucleotide, two nucleotides, or three nucleotides.

27. The method of any one of the above claims, the primer does not comprise a 5′ phosphate group.

28. The method of any one of the above claims, the sequencing adapter comprises a second barcode.

29. The method of any one of the above claims, wherein the base modification comprises a 5-methylcytosine (5mC), a 5-hydroxymethylcytosine (5hmC), a N 6-methyladenine (6 mA), a 7-methylguanine (m7G), 2′-O-methylation (2′-O-Methyl), or a bromodeoxyuridine (BrdU).

30. The method of any one of the above claims, wherein the method does not comprise bisulfite conversion or amplification of the target polynucleotide.

31. The method of any one of the above claims, wherein the method does not comprise reduced representation bisulfite sequencing (RRBS) or reduced representation methylation sequencing (RRMS).

32. The method of any one of the above claims, wherein the first ligase or the second ligase is a T4 DNA ligase, a Quick ligase, a T3 ligase, a T7 ligase, or a Tag ligase.

33. The method of any one of the above claims, wherein the first polymerase is a Taq polymerase, a BST polymerase, a sulfolobus polymerase, a therminator polymerase, a Klenow polymerase, a deep vent polymerase.

34. The method of any one of the above claims, wherein sequencing is performed on a sequencer capable of identifying modified bases.

35. The method of any one of the above claims, wherein the base modification in the target polynucleotide is retained until sequencing.

36. The method of any one of the above claims, wherein the method enriches the base modification in the target polynucleotide between about 300-fold to about 2400-fold compared to whole genome sequencing.
